SEQUENCE LISTING (66 sequences)

SEQ ID NO: 1 - DNA, 20 bp, Artificial Sequence
  Synthetic Polynucleotide
ggactachvgggtatctaat

SEQ ID NO: 2 - DNA, 19 bp, Artificial Sequence
  Synthetic Polynucleotide
  misc_feature (14)..(14): n is a, c, g, t or u
cgacrrccatgcancacct

SEQ ID NO: 3 - DNA, 15 bp, Artificial Sequence
  Synthetic Polynucleotide
  misc_feature (6)..(6): n is a, c, g, t or u
  misc_feature (9)..(9): n is a, c, g, t or u
gggttncgntcgttg

SEQ ID NO: 4 - DNA, 15 bp, Artificial Sequence
  Synthetic Polynucleotide
  misc_feature (14)..(14): n is a, c, g, t or u
aytgggydtaaagng

SEQ ID NO: 5 - DNA, 16 bp, Artificial Sequence
  Synthetic Polynucleotide
  misc_feature (12)..(12): n is a, c, g, t or u
gtgccagcmgcngcgg

SEQ ID NO: 6 - DNA, 20 bp, Artificial Sequence
  Synthetic Polynucleotide
  misc_feature (8)..(8): n is a, c, g, t or u
bgyctwanrcatgcaagtyg

SEQ ID NO: 7 - DNA, 20 bp, Artificial Sequence
  Synthetic Polynucleotide
caatggrsgvrasyctgahs

SEQ ID NO: 8 - DNA, 21 bp, Artificial Sequence
  Synthetic Polynucleotide
gactachvgggtatctaatcc

SEQ ID NO: 9 - DNA, 18 bp, Artificial Sequence
  Synthetic Polynucleotide
ccgacggtgagrgrygaa

SEQ ID NO: 10 - DNA, 19 bp, Artificial Sequence
  Synthetic Polynucleotide
  misc_feature (2)..(2): n is a, c, g, t or u
  misc_feature (18)..(18): n is a, c, g, t or u
cnacgcgaagaaccttanc

SEQ ID NO: 11 - DNA, 20 bp, Artificial Sequence
  Synthetic Polynucleotide
attcyggttgatccygscrg

SEQ ID NO: 12 - DNA, 17 bp, Artificial Sequence
  Synthetic Polynucleotide
waggaggtratccadcc

SEQ ID NO: 13 - DNA, 18 bp, Artificial Sequence
  Synthetic Polynucleotide
  misc_feature (4)..(4): n is a, c, g, t or u
tacnvgggtatctaatcc

SEQ ID NO: 14 - DNA, 18 bp, Artificial Sequence
  Synthetic Polynucleotide
ggmttagatacccbdgta

SEQ ID NO: 15 - DNA, 19 bp, Artificial Sequence
  Synthetic Polynucleotide
gaattgacggggrcccgca

SEQ ID NO: 16 - DNA, 19 bp, Artificial Sequence
  Synthetic Polynucleotide
atacgcgargaaccttacc

SEQ ID NO: 17 - DNA, 19 bp, Artificial Sequence
  Synthetic Polynucleotide
ctaccrgggtatctaatcc

SEQ ID NO: 18 - DNA, 19 bp, Artificial Sequence
  Synthetic Polynucleotide
  misc_feature (14)..(14): n is a, c, g, t or u
cgacgaccatgcancacct

SEQ ID NO: 19 - DNA, 20 bp, Artificial Sequence
  Synthetic Polynucleotide
ccgtcaattcmtttgagttt

SEQ ID NO: 20 - DNA, 20 bp, Artificial Sequence
  Synthetic Polynucleotide
  misc_feature (2)..(2): n is a, c, g, t or u
  misc_feature (4)..(4): n is a, c, g, t or u
tnanacatgcaagtcgrrcg

SEQ ID NO: 21 - DNA, 18 bp, Artificial Sequence
  Synthetic Polynucleotide
tachvgggtatctaakcc

SEQ ID NO: 22 - DNA, 20 bp, Artificial Sequence
  Synthetic Polynucleotide
tcacgrcacgagctgacgac

SEQ ID NO: 23 - DNA, 16 bp, Artificial Sequence
  Synthetic Polynucleotide
gyaacgagcgcaaccc

SEQ ID NO: 24 - DNA, 16 bp, Artificial Sequence
  Synthetic Polynucleotide
gggttgcgctcgttrc

SEQ ID NO: 25 - DNA, 18 bp, Artificial Sequence
  Synthetic Polynucleotide
tagatacccssgtagtcc

SEQ ID NO: 26 - DNA, 20 bp, Artificial Sequence
  Synthetic Polynucleotide
  misc_feature (3)..(3): n is a, c, g, t or u
ctnygtmttaccgcggctgc

SEQ ID NO: 27 - DNA, 17 bp, Artificial Sequence
  Synthetic Polynucleotide
crrcacgagctgacgac

SEQ ID NO: 28 - DNA, 17 bp, Artificial Sequence
  Synthetic Polynucleotide
cctacgggrsgcagcag

SEQ ID NO: 29 - DNA, 18 bp, Artificial Sequence
  Synthetic Polynucleotide
gwattaccgcggckgctg

SEQ ID NO: 30 - DNA, 19 bp, Artificial Sequence
  Synthetic Polynucleotide
  misc_feature (14)..(14): n is a, c, g, t or u
cgacaaccatgcancacct

SEQ ID NO: 31 - DNA, 19 bp, Artificial Sequence
  Synthetic Polynucleotide
  misc_feature (9)..(9): n is a, c, g, t or u
ctaaccgangaacctyacc

SEQ ID NO: 32 - DNA, 15 bp, Artificial Sequence
  Synthetic Polynucleotide
acgggcggtgtgtrc

SEQ ID NO: 33 - DNA, 16 bp, Artificial Sequence
  Synthetic Polynucleotide
  misc_feature (15)..(15): n is a, c, g, t or u
acgacagccatgcanc

SEQ ID NO: 34 - DNA, 16 bp, Artificial Sequence
  Synthetic Polynucleotide
gacgggcggtgtgtrc

SEQ ID NO: 35 - DNA, 16 bp, Artificial Sequence
  Synthetic Polynucleotide
cgcgaagaaccttacc

SEQ ID NO: 36 - DNA, 16 bp, Artificial Sequence
  Synthetic Polynucleotide
gcatggcygycgtcag

SEQ ID NO: 37 - DNA, 16 bp, Artificial Sequence
  Synthetic Polynucleotide
ctgacgrcrgccatgc

SEQ ID NO: 38 - DNA, 26 bp, Artificial Sequence
  Synthetic Polynucleotide
ggactaccagggtatctaatcctgtt

SEQ ID NO: 39 - DNA, 18 bp, Artificial Sequence
  Synthetic Polynucleotide
cgtcaattcmtttgagtt

SEQ ID NO: 40 - DNA, 18 bp, Artificial Sequence
  Synthetic Polynucleotide
cagcmgccgcggtaatwc

SEQ ID NO: 41 - DNA, 18 bp, Artificial Sequence
  Synthetic Polynucleotide
actcaaakgaatwgacgg

SEQ ID NO: 42 - DNA, 20 bp, Artificial Sequence
  Synthetic Polynucleotide
caatggrsgcrasyctgahs

SEQ ID NO: 43 - DNA, 20 bp, Artificial Sequence
  Synthetic Polynucleotide
  misc_feature (8)..(8): n is a, c, g, t or u
bgyctwanrcatgcaagtyg

SEQ ID NO: 44 - DNA, 15 bp, Artificial Sequence
  Synthetic Polynucleotide
  misc_feature (14)..(14): n is a, c, g, t or u
aytgggydtaaagng

SEQ ID NO: 45 - DNA, 16 bp, Artificial Sequence
  Synthetic Polynucleotide
  misc_feature (12)..(12): n is a, c, g, t or u
gtgccagcmgcngcgg

SEQ ID NO: 46 - DNA, 20 bp, Artificial Sequence
  Synthetic Polynucleotide
ggactachvgggtatctaat

SEQ ID NO: 47 - DNA, 19 bp, Artificial Sequence
  Synthetic Polynucleotide
  misc_feature (14)..(14): n is a, c, g, t or u
cgacrrcaatgcancacct

SEQ ID NO: 48 - DNA, 15 bp, Artificial Sequence
  Synthetic Polynucleotide
  misc_feature (6)..(6): n is a, c, g, t or u
  misc_feature (9)..(9): n is a, c, g, t or u
gggttncgntcgttg

SEQ ID NO: 49 - DNA, 43 bp, Artificial Sequence
  Synthetic Polynucleotide
cgcatacctctcccaaatctccgtttactgcacctaatcacct

SEQ ID NO: 50 - DNA, 41 bp, Artificial Sequence
  Synthetic Polynucleotide
cgcatacctctccaatctccgtttactgcacctaatcacct

SEQ ID NO: 51 - DNA, 30 bp, Artificial Sequence
  Synthetic Polynucleotide
aaatttgccgactcgcataggtctgtgata

SEQ ID NO: 52 - DNA, 41 bp, Artificial Sequence
  Synthetic Polynucleotide
aggtgattaggtgcagtaaacggagattggagaggtatgcg

SEQ ID NO: 53 - DNA, 20 bp, Artificial Sequence
  Synthetic Polynucleotide
gaccccaaaatcagcgaaat

SEQ ID NO: 54 - DNA, 24 bp, Artificial Sequence
  Synthetic Polynucleotide
tctggttactgccagttgaatctg

SEQ ID NO: 55 - DNA, 24 bp, Artificial Sequence
  Synthetic Polynucleotide
  misc_feature (1)..(1): FAM
  misc_feature (24)..(24): BHQ1
accccgcattacgtttggtggacc

SEQ ID NO: 56 - DNA, 24 bp, Artificial Sequence
  Synthetic Polynucleotide
  misc_feature (1)..(1): FAM
  misc_feature (9)..(9): ZEN
  misc_feature (24)..(24): 3IABkFQ
accccgcattacgtttggtggacc

SEQ ID NO: 57 - DNA, 20 bp, Artificial Sequence
  Synthetic Polynucleotide
ttacaaacattggccgcaaa

SEQ ID NO: 58 - DNA, 18 bp, Artificial Sequence
  Synthetic Polynucleotide
gcgcgacattccgaagaa

SEQ ID NO: 59 - DNA, 22 bp, Artificial Sequence
  Synthetic Polynucleotide
  misc_feature (1)..(1): FAM
  misc_feature (22)..(22): BHQ1
acaatttgcccccagcgcttca

SEQ ID NO: 60 - DNA, 23 bp, Artificial Sequence
  Synthetic Polynucleotide
  misc_feature (1)..(1): FAM
  misc_feature (9)..(9): ZEN
  misc_feature (23)..(23): 3IABkF
acaatttgcccccagcgcttcag

SEQ ID NO: 61 - DNA, 19 bp, Artificial Sequence
  Synthetic Polynucleotide
agatttggacctgcgagcg

SEQ ID NO: 62 - DNA, 20 bp, Artificial Sequence
  Synthetic Polynucleotide
gagcggctgtctccacaagt

SEQ ID NO: 63 - DNA, 23 bp, Artificial Sequence
  Synthetic Polynucleotide
  misc_feature (1)..(1): FAM
  misc_feature (23)..(23): BHQ-1
ttctgacctgaaggctctgcgcg

SEQ ID NO: 64 - DNA, 23 bp, Artificial Sequence
  Synthetic Polynucleotide
  misc_feature (9)..(9): ZEN
  misc_feature (23)..(23): 3IABkFQ
ttctgacctgaaggctctgcgcg

SEQ ID NO: 65 - DNA, 20 bp, Artificial Sequence
  Synthetic Polynucleotide
  misc_feature (1)..(1): Bio
  misc_feature (8)..(8): n is a, c, g, t or u
bgyctwanrcatgcaagtyg

SEQ ID NO: 66 - DNA, 40 bp, Artificial Sequence
  Synthetic Polynucleotide
ctaccagggtatctaatcctgtttgctccccacgctttcg

(19) United States
(12) Patent Application Publication
(10) Pub. No.: US 2022/0364158 A1
(43) Pub. Date: Nov. 17, 2022

(54) METHODS AND DEVICES FOR IDENTIFYING PATHOGENS AND ANTIBODIES AND TREATMENT DEVICE THEREFORE

(71) Applicant: THE BOARD OF TRUSTEES OF THE UNIVERSITY OF ILLINOIS, Urbana, IL (US)

(72) Inventors: Yajing Song, Peoria, IL (US); Peter Gyarmati, Peoria, IL (US)

(21) Appl. No.: 17/621,989

(22) PCT Filed: Jun. 25, 2020
(86) PCT No.: PCT/US20/39684

§ 371 Date: Dec. 22, 2021

Related U.S. Application Data
(60) Provisional application No. 62/867,015, filed on Jun. 26, 2019.
(60) Provisional application No. 62/866,605, filed on Jun. 25, 2019.

(51) Int. Cl. C12Q 1/6816; C12Q 1/6834; C12Q 1/70

(52) U.S. Cl. CPC C12Q 1/6816; C12Q 1/6834; C12Q 1/701

(57) ABSTRACT

Disclosed herein are point-of-care (POC) diagnostic devices comprising a fibrous carrier, e.g., filter paper, having one or more capture probes configured to display one or more visual outputs indicating the presence of a pathogen in a sample. Also provided herein are methods of using POC diagnostic devices to detect the presence of a pathogen in a sample, e.g., to detect the presence of bacteria in blood.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “55706_Seqlisting.txt”, which was created on Jun. 25, 2020 and is 16,089 bytes in size. The subject matter of the Sequence Listing is incorporated herein in its entirety by reference.

### BACKGROUND

Processes to identify pathogens are important in a number of settings, including food manufacture, pharmaceutical manufacture, and hospitals to name a few. For example, bloodstream infection (BSI) is a life-threatening condition characterized by the presence of pathogens in the blood. It is associated with increased morbidity and mortality, and has to be treated promptly as mortality increases with every hour of delayed treatment. Therefore, rapid and sensitive identification and diagnosis of BSI is an essential need in hospitals.

The current routine diagnostic method for BSI is blood culture can only detect culturable pathogens and takes several days to obtain results. The 16S rRNA gene is present in all bacteria and is commonly used as a target for universal bacterial detection in rapid molecular assays such as PCR. However, molecular detection of the 16S gene is hampered by the large amount of human DNA found in blood samples, making diagnostic results a specific and less sensitive.

### SUMMARY

Rapid bedside detection is crucial for patients with life-threatening infection to immediately initiate an accurate case management. However, even the best diagnostic method cannot meet this need currently. Described herein are surface-functionalization systems of cellulose filter paper by using glutaric anhydride, N-hydroxysuccinimide, N, N′-Dicyclohexylcarbodiimide and methanol. Both synthetic oligonucleotides and bacterial genomic DNA can be targeted and detected in accordance with the principles of the present disclosure. Systems constructed in accordance with the principles herein can be used in point of care (POC) devices configured to produce a clear, regular and visible signal rapidly by naked eye, which can also be analyzed quantitatively. Bacterial detection can be accomplished by using 16S rDNA probes on the activated paper surface for universal bacterial diagnosis. The methods described herein are stable and repeatable, while the devices can rapidly detect bacterial, viral, and fungal pathogens.

For example, in patients with leukemia, blood stream infection is a major cause of death. Round time from accurate pathogen detection in blood to specific antibiotics treatment is negatively correlated with survival rate. Therefore, rapid detection of pathogens is essential for efficient and effective treatment. The limit of current routine detection (blood culture) is time-consuming, however a method constructed in accordance with the principles herein can be applied in rapid and specific DNA detection of pathogens in bloodstream infection.

In one aspect, the disclosure provides point of care (POC) diagnostic devices for identifying a target nucleic acid sequence in a sample, comprising: a fibrous carrier comprising functionalized fibers; and one or more capture probes bound to one or more of the functionalized fibers in one or more first discrete locations of the fibrous carrier, the one or more capture probes being capable of selectively binding with the target nucleic acid sequence. In embodiments, the device can include multiple distinct capture probes capable of binding to more than one target nucleic acid sequences, each of the capture probes of a given type being arranged in a single first discrete location. Such devices can allow for detecting of multiple potential target nucleic acid sequences in a sample. For example, a sample suspected to be contaminated can be tested on a device in accordance with the disclosure having two or more distinct capture probes types to allow for detection of two or more distinct pathogens. For example, the devices of the disclosure can have capture probes in one discrete location selective to bind to a target nucleic acid sequence of bacterium and capture probes in another discrete location selective to bind to a target nucleic acid sequence of viruses.

In one aspect, the disclosure provides point of care (POC) diagnostic devices for identifying a target nucleic acid sequence in a sample, comprising: a fibrous carrier comprising functionalized fibers; and one or more capture probes bound to one or more of the functionalized fibers in one or more first discrete locations of the fibrous carrier, the one or more capture probes being capable of selectively binding with the target nucleic acid sequence and one or more control probes bound to one or more of the functionalized fibers in one or more second discrete locations of the fibrous carrier, and one or more indicia disposed on the fibrous carrier to identify the first and second discrete locations.

In another aspect, the disclosure provides methods for making point of care (POC) diagnostic devices disclosed herein, comprising the steps of: (a) treating a fibrous carrier with one or more reagents to functionalize fibers of the fibrous carrier with a nucleic acid binding moiety; (b) binding a capture probe to one or more of the functionalized fibers in a first discrete location of the fibrous carrier, the nucleic acid binding moiety binding the capture probe to the one or more functionalized fibers; and (c) applying a control to a second discrete location of the fibrous carrier.

In another aspect, the disclosure provides methods for detecting the presence of a pathogen in a sample, the method comprising: (a) contacting the sample comprising or suspected of comprising a target nucleic acid sequence from the pathogen with a visual label under conditions to bind the visual label to a target nucleic acid sequence thereby providing a labeled sample; (b) contacting a POC diagnostic device disclosed herein with the labeled sample, wherein upon contact the target nucleic acid sequence if present binds to one or more of the capture probes congregating the visual label attached to the nucleic acid sequence of the pathogen in the first discrete location and generating a visual output; and (c) washing the device to remove unbound portions of the sample.

In another aspect, the disclosure provides kits for determining the presence of a pathogen in a sample comprising: a detection device, comprising: a fibrous carrier comprising functionalized fibers; and one or more capture probes bound to one or more of the functionalized fibers in one or more first discrete locations of the fibrous carrier, the one or more capture probes being capable of selectively binding with the target nucleic acid sequence; and one or more control probes bound to one or more of the functionalized fibers in one or more second discrete locations of the fibrous carrier; a visual label for labeling the sample; and instructions for labeling a sample with the visual label and contacting the sample with the detection device to transport the sample through the fibrous carrier and expose the target nucleic acid sequence, if present, to the one or more capture probes.

### BRIEF DESCRIPTION OF THE DRAWINGS

FIG. 1 illustrates primer selection using gel electrophoresis on a 2% agarose gel and shows amplification of E. coli (EC) and human genomic DNA (HU) with selected primer pairs. The 341-803 and 530-803 primer pairs resulted in misamplifications with human genomic DNA, while the 363-806 primers produced secondary PCR products. All other primer pairs resulted in specific amplification of the 16S rRNA gene. The concentrations of the samples were: 55 ng/μl for EC (165 ng total) and 11 ng/μl (33 ng total) for HU.

FIG. 2 illustrates a bar graph of primer sensitivities without producing false amplifications products using standard amount of human genome with serial dilutions of E. coli (10⁻¹-10⁻⁴).

FIG. 3 is a schematic showing the layout of a printing array as used in Example 2 from position 1 to position 4 (p1-p4).

FIG. 4 is a set of graphs showing an overview of the functionalization methods. FIG. 4A: Investigation of eight functionalized methods on the surface of filter paper (FP). FP is a blank control. GND-Methanol-GND is a positive control. FIG. 4B: There is a significant difference between the signal intensities of GND-Methanol-GND and 10% PAM-GND. FIG. 4C: Evaluation of nine methods using APTS or PAMAM with combinations of GND or PDITC or GND and PDITC. PAM-GND is a positive control. Detailed description of the methods are found in Example 2.

FIG. 5 is a photograph showing visual detection comparison between the methods of PAMAM-PDITC, PAMAM-PDITC-PAMAM-PDITC and PAMAM-PDITC-PAMAM-GND.

FIG. 6 is a series of graphs showing parameter optimization using the PAMAM-PDITC-PAMAM-PDITC functionalization method. FIG. 6A: Comparison of PDITC concentration. FIG. 6B: Comparison of volume of PAMAM in methanol. FIG. 6C: Carbon spacer arm comparison. FIG. 6D: Comparison of “APT₂-PBF” intensities from different solutions containing PAMAM dendrimer.

FIG. 7 is a series of graphs showing optimization of the amounts of: FIG. 7A: printed probes (20 μM), FIG. 7B: target DNAs, and FIG. 7C: magnetic bead volume.

FIG. 8 is a comparison of the different systems for functionalization of the filter paper surface.

FIG. 9 is a series of graphs showing evaluations of: FIG. 9A: control position (A, B, T represent APT₂W/O, 1×PBF, and TID), FIG. 9B: wash method (wash methods from 1 to 5 are 4×SSC buffer containing 0.01% SDS, 2×SSC buffer containing 0.01% SDS, heated deionized water, room-temperature deionized water and 1×SSC buffer containing 0.01% SDS), FIG. 9C: solutions that the controls dissolved in, and FIG. 9D: carbon spacer length.

FIG. 10 is a series of graphs showing optimizations of: FIG. 10A: probe volume, FIG. 10B: target amount, and FIG. 100: bead volume.

FIG. 11 is a series of graphs and a photograph showing an overview of 16S rRNA gene Detection. FIG. 11A: Different concentrations of NHS/DCC in DMSO are evaluated while activating filter paper. FIG. 11B: Visual detection of Bacterial DNA on site.

FIG. 110: Three volumes of printed probe are compared.

FIG. 12 is a schematic presentation of the functionalization of filter paper, detection, and image analysis.

FIG. 13 is a schematic showing an example timeline of work flow.

FIG. 14 is a schematic showing a test strip and fluid sample.

FIG. 15 is a photograph showing an example of small fluid sample size.

### DETAILED DESCRIPTION

Paper-based immunoassays have been used for e.g., over-the-counter pregnancy tests; however, such immunoassays are based on protein detection. The devices and methods disclosed herein are useful for detecting DNA, which provides a higher level of multiplexing than protein-based immunoassays, e.g., the devices and methods disclosed herein can detect four or more targets simultaneously. In addition, commercial assays such as pregnancy tests employ nitrocellulose. The devices and methods disclosed herein employ fibrous carriers such as cellulose filter paper, which provides a larger 3D structure, higher flow rate, and lower cost compared with nitrocellulose.

Currently, DNA detection typically employs devices based on glass surfaces that require specialized equipment and time to manufacture. In contrast, the devices and methods disclosed herein employ functionalized fibrous carriers, e.g., functionalized filter paper, which has a 3D structure to increase printed probe density and flow rate of sample solutions containing targets. These features provide a rapid and cost-efficient detection.

Visual labels disclosed herein can include e.g., superparamagnetic beads. The use of superparamagnetic beads in the devices and methods disclosed herein confers several advantages, including but not limited to, allowing for efficient collection of bead-labeled targets in a sample in a short period of time using a magnet; providing a color signal against a white filter paper surface when bead-labeled targets bind to capture probes disposed therein; and providing a curved surface that enables higher target-loading capacity than a flat surface. These features can be particularly advantages in point of care devices such as disclosed herein which offer visual detection capability.

In accordance with the embodiments, a point-of-care detection device or pathogen detection device is provided through the introduction of surface chemistries in filter paper. One or more different chemicals can be used to activated the surface of the fibrous carrier, as described in detail below. Specific biomarkers can then be detected on the functionalized fibrous carrier through interactions such as binding interactions, which can lead to a detectable signal. Devices and methods of the disclosure advantageously use fibrous based carriers, such as cellulose filter paper, which is a readily available commodity. Cellulose filter paper mainly consists of cellulose fiber and provide porosity and particle retention properties which can be advantage in the devices and methods of the disclosure. These properties allow filter paper to functionalized with chemicals to bind DNA probes. Complementary DNA can be labeled with, for example, iron-oxide beads or other suitable label, which can provide a visible indication of successful binding (between the printed and complementary DNA). Such visible indication can be observable by the naked eye, without the need to use instrumentation. The sample can be processed through the filter paper by capillary force due to its hydrophilic feature and porous structure. Furthermore, the three dimensional space formed by an interwoven structure of fiber or carrier makes the high density of nanomolecules immobilized on the functionalized filter paper.

Advantageously, the methods and devices of the disclosure can provide rapid, instrument-free detection of DNA. The method is cost-efficient, easy to operate and free of geographical restrictions based on equipment to analyze the result as is required with the current systems.

Methods and devices of the disclosure can provide point-of-care test of a number of pathogens, such as blood stream infection, and can increase the survival rate of the patients with leukemia for example. Methods and devices of the disclosure can also provide universal bacterial detection.

Abbreviations herein include: POC, point of care; PAMAM, polyamidoamine; PDITC, p-Phenylene diisothiocyanate; LFAs, Lateral Flow immunoassays; FP, Filter paper; DMSO, Dimethyl sulfoxide; APTS, 3-aminopropyltriethoxysilane; GA, glutaric anhydride; NHS, N-hydroxysuccinimide; DCC, N,N′-Dicyclohexylcarbodiimide; DMF, N,N-dimethylformamide; GND, the combination of three chemicals: GA, NHS and DCC.

Provided herein are point of care (POC) diagnostic devices comprising a fibrous carrier having one or more capture probes configured to display one or more visual outputs indicating the presence of a bacterium, a virus, a fungus, or combinations thereof in a sample. In some cases, the fibrous carrier comprises paper. In some cases, the fibrous carrier comprises filter paper. In some cases, the fibrous carrier comprises cellulose filter paper. Also provided are point of care (POC) diagnostic devices for identifying a target nucleic acid sequence in a sample, comprising: a fibrous carrier comprising functionalized fibers; and one or more capture probes bound to one or more of the functionalized fibers in one or more first discrete locations of the fibrous carrier, the one or more capture probes being capable of selectively binding with the target nucleic acid sequence and one or more control probes bound to one or more of the functionalized fibers in one or more second discrete locations of the fibrous carrier, and one or more indicia disposed on the fibrous carrier to identify the first and second discrete locations.

In some cases, the filter paper is highly porous. As used herein, the term “highly porous” means that the filter paper contains many or relatively large pores that allow for the passage of material through the paper, e.g., pores that retain particles larger than about 11 μm. For example, filter paper used herein can retain particles larger than about 5 μm, larger than about 6 μm, larger than about 7 μm, larger than about 8 μm, larger than about 9 μm, larger than about 10 μm, larger than about 11 μm, larger than about 12 μm, larger than about 20 μm, larger than about 25 μm, or larger than about 50 μm. In some cases, the filter paper can retain particles larger than about 5 μm to about 10 μm. In some cases, the filter paper can retain particles larger than about 8 μm to about 10 μm. In some cases, filter paper used herein can retain particles larger than about 5 μm. In some cases, filter paper used herein can retain particles larger than about 6 μm. In some cases, filter paper used herein can retain particles larger than about 7 μm. In some cases, filter paper used herein can retain particles larger than about 8 μm. In some cases, filter paper used herein can retain particles larger than about 9 μm. In some cases, filter paper used herein can retain particles larger than about 10 μm. In some cases, filter paper used herein can retain particles larger than about 11 μm. In some cases, filter paper used herein can retain particles larger than about 12 μm. In some cases, filter paper used herein can retain particles larger than about 20 μm. In some cases, filter paper used herein can retain particles larger than about 25 μm. In some cases, filter paper used herein can retain particles larger than about 50 μm.

Thus, in accordance with the principles herein, embodiments of a point of care (POC) diagnostic device can take a number of forms, even beyond the basic examples shown and discussed herein. For example, in an embodiment a POC device constructed in accordance with the principles herein can include: a single fibrous carrier configured to receive and transport a fluid sample to one or more embedded capture probes, each of the one or more embedded capture probes configured to visually display one or more outputs indicating rapid universal detection of a bacterium and/or a fungus, and one or more specific target bacteria and/or one or more specific target fungi and/or one or more specific viruses present in the fluid sample.

In certain embodiments the fibrous carrier of the POC diagnostic device can be further defined by a filter paper; and/or the fluid sample can be further defined by a small sample size in the range of 0.01 ml-0.5 ml; and/or the fluid sample can include at least one of blood, urine, saliva, breast milk, mucus, pus, sweat, tears, CSF, semen, secretions, serum, plasma or bronchoalveolar lavage fluid; and/or the fluid sample can contains a bodily fluid; and/or the fluid sample can contain fluid used in the manufacturing of pharmaceutical or food products; and/or the fluid sample contains bottled water; and/or the fluid sample contains metalworking fluid, coolant or potable water.

In certain embodiments, a POC diagnostic device can be configured to identify antimicrobial resistance genes in the fluid sample. In some embodiments a POC diagnostic device can be configured so that one or more of DNA and/or protein components in the fluid sample are detectable and identifiable from the fluid sample received via the single fibrous carrier.

A POC diagnostic device constructed in accordance with the principles herein can include one or more embedded capture probes, each capture probe configured to display a visual output indicating rapid detection of antimicrobial resistance genes. In still other embodiments antimicrobial resistance genes in the fluid sample are detectable and identifiable from the fluid sample received via the single fibrous carrier at the embedded capture target. In certain embodiments antimicrobial resistance genes in the fluid sample are detectable and identifiable from a smaller fluid sample than currently required to detect antimicrobial resistance genes in the laboratory.

POC diagnostic devices can produce one or more outputs, each output having a variation in intensity based on levels of the one or more specific target bacteria or the one or more specific target fungi or the one or more specific target viruses present in the fluid sample, such that an image of the single fibrous carrier can indicate categories and quantity of pathogens, which can further guide treatment of a fluid sample source.

POC diagnostic devices can produce one or more outputs each having a variation in intensity based on levels of the one or more specific target bacteria or the one or more specific target fungi or the one or more specific target viruses present in the fluid sample, such that an image of the single fibrous carrier can indicate categories and quantity of pathogens in a human patient or a sick animal, which can further guide treatment and case management of the patient or the sick animal.

POC diagnostic devices can produce one or more outputs providing an indication of an intensity level of a bacterial and/or viral and/or fungal concentration in the fluid sample.

The one or more outputs can be generated via a chemical reaction between the fluid sample labeled with beads and suitable probe material present in the embedded capture probe, and/or the one or more outputs can be generated via a chemical reaction between the fluid sample and color generating components.

In some cases, the output can be generated via a chemical reaction between an amplicon labeled with superparamagnetic beads and suitable probe material present in the embedded capture probe. In some cases, the amplicon can be generated via PCR amplification of a sample. In some cases, the amplicon can be a DNA amplicon. In some cases, the amplicon can be an RNA amplicon. The PCR amplification of a sample can be carried out via methods known to the skilled artisan, e.g., as described in Song et al., Microchimica Acta (2019) 186: 642, incorporated herein by reference.

A POC diagnostic device can further include an orientation component to aid in confirming the location of the one or more embedded capture probes on the single fibrous carrier. The location of one or more embedded capture probes on a single fibrous carrier can be determined by the position of the one or more embedded capture probes on the single fibrous carrier, and/or an orientation component can be provided that includes at least one of printed text and other indicia. In still other embodiments, the location of an embedded capture probes can include any of the above identification options and/or an offset to the paper can determine the capture probe location.

In some POC diagnostic devices, the one or more embedded capture probes further include activateable treatments embedded in the one or more embedded capture probes that release in response to rapid detection of the one or more specific target bacteria and/or the one or more specific target fungi and/or the one or more specific target viruses present in the fluid sample.

Provided herein are POC diagnostic devices comprising functionalized fibrous carriers. For ease of reference, fibrous carriers are referred to throughout the disclosure by way of one example, a filter paper. Use of fibrous carriers other than filter paper are also contemplated herein and reference to filter paper in the descriptions herein should not be considered limiting the type of fibrous carrier. As used herein, the term “functionalized” fibrous carriers refers to fibrous carriers which have been treated with reagents disclosed herein in order to attach one or more additional species to the carrier. For example, this includes treatment of filter paper with reagents in order to attach a nucleic acid-binding moiety.

In some cases, the POC devices disclosed herein have a fibrous carrier comprising activated functional groups to which one or more capture probes are selectively attached to the functional groups in a first discrete location of the fibrous carrier, with one or more controls optionally selectively attached to the functional groups in discrete locations adjacent to the capture probes. In some cases, the devices have one or more controls optionally selectively attached to the functional groups in discrete locations adjacent to the capture probes. In some cases, the devices can have an array of regions comprising multiple probes for detecting different targets. In some cases, the devices can have an array of regions comprising probes capable of detecting two or more of bacteria, viruses, and fungi. In some cases, the devices can have an array of regions comprising probes capable of detecting one or more types of bacteria. In some cases, the devices can have one or more controls adjacent to each type of probe. In some cases, the device can comprise multiple controls chosen based on the type or types of probe used. In some cases, the control can be blank filter paper. In some cases, the control can be sodium phosphate buffer (PBF), TID, or APT₂WO. In some cases, the control can be sodium phosphate buffer (PBF). In some cases, the control can be TID. In some cases, the control can be APT₂WO. In some cases, the control can be an artificial oligonucleotide or known pathogen DNA.

In some cases, functionalized fibrous carrier comprises a binding moiety. As used herein, the term “binding moiety” refers to a functional group or portion of a molecule that binds to a desired class of target molecule. In some cases, the binding moiety can be a nucleic acid binding moiety. In some cases, the binding moiety can be a DNA binding moiety. In some cases, the binding moiety can be an RNA binding moiety. In some cases, the binding moiety can be a protein binding moiety.

In some cases, the nucleic acid binding moiety can be produced by treating the filter paper with polyamidoamine (PAMAM) dendrimer and p-phenylene diisothiocyanate (PDITC). In some cases, the nucleic acid binding moiety can be produced by treating the filter paper with glutaric anhydride (GA), N-hydroxysuccinimide, N, N′-dicyclohexylcarbodiimide, and methanol.

In some cases, the devices disclosed herein can be made by activating the fibrous carrier with one or more of polyamidoamine (PAMAM) dendrimer and p-phenylene diisothiocyanate (PDITC) or glutaric anhydride (GA), N-hydroxysuccinimide, N, N′-dicyclohexylcarbodiimide, and methanol, followed by binding a capture probe to the activated fibrous carrier in a discrete location. In some cases, a control is bound to the fibrous carrier in a discrete location adjacent to the discrete location of the capture probe.

Without wishing to be bound by theory, fibrous carriers such as filter paper often comprise cellulose, which has an open, three-dimensional structure bearing terminal hydroxyl groups. Again without wishing to be bound by theory, these hydroxyl groups can be transformed into other functional groups, such as carboxylic acid groups, by appropriate reagents (e.g., glutaric anhydride (“GA”). Again without wishing to be bound by theory, linker reagents can be used to physically distance the functional groups, such as carboxylic acid groups, from the surface of the fibrous carrier, e.g., by using aminopropyl triethoxysilane (APTS), and/or to increase the surface area available to capture targets, e.g., by using polyamidoamine (“PAM” or “PAMAM”) dendrimer. If the linker has an amine functional group, it can be further functionalized by treatment with GA to produce carboxylic acid groups on the surface. Without wishing to be bound by theory, the carboxylic acid functional groups can be converted to nucleic acid binding moieties by treatment with appropriate reagents. Non-limiting examples of nucleic acid binding moieties that can be produced by the methods described herein include active esters formed by treatment of the carboxylic acid-functionalized fibrous carrier with e.g., carbodiimide or diisothiocyanate reagents, such as N,N′-dicyclohexylcarbodiimide (“DCC”) in the presence of N-hydroxysuccinimide (“NHS”), or p-phenylene diisothiocyanate (“PDITC”). Again without wishing to be bound by theory, these active esters can react with appropriate moieties on the captive probes, e.g., with amine groups present in proteins or aminated DNA.

Functionalization of fibrous carriers can be carried out by successive treatment of the carrier with reagents in a defined order. For example, cellulose filter paper can be treated successively with GA, NHS, and DCC to produce active esters on the paper; this functionalization sequence is also called “GND” herein. As used herein, “GND-methanol” refers to functionalization of a fibrous carrier by successive treatment with GA, NHS, and DCC, followed by fixation with methanol. Without wishing to be bound by theory, fixation with methanol can be advantageous because it washes away unused reagents while preserving the cellulose fibers and their dimensions in the filter paper.

After functionalization as described herein, the fibrous carriers can be treated with a capture probe, e.g., to embed the capture probe in the fibrous carrier. Provided herein are POC diagnostic devices comprising a capture probe capable of binding to one or more pathogen targets. In some cases, the capture probe can be embedded in the fibrous carrier. In some cases, the capture probe is embedded in filter paper. In some cases, the capture probe can be a nucleic acid. In some cases, the capture probe can be synthetic oligonucleotides, genomic DNA, or genomic RNA. In some cases, the capture probe can be synthetic oligonucleotides. In some cases, the capture probe can be genomic DNA. In some cases, the capture probe can be genomic RNA.

In some cases, treating the fibrous carrier with a capture probe comprises pipetting a solution comprising a capture probe onto the fibrous carrier. In some cases, pipetting the solution comprises manual pipetting. In some cases, pipetting the solution comprises automatic pipetting. In some cases, treating the fibrous carrier with a capture probe comprises printing a solution onto the fibrous carrier. Other printing techniques, such as inkjet printing, can also be used herein in accordance with known printing techniques

In some cases, the capture probe can be a primer which amplifies a nucleic acid. In some cases, the capture probe can be bacterial genomic DNA. In some cases, the capture probe can be a primer which amplifies a bacterial genomic DNA. It will be understood that primers which amplify a target nucleic acid, such as primers which amplify a bacterial genomic DNA, include primers which can hybridize with the target nucleic acid. In some cases, the primer can amplify a bacterial genomic DNA without cross-reacting with a human genomic DNA. In some cases, the capture probe can be a primer which amplifies a 16S rRNA gene. In some cases, the capture probe can be a primer as recited in Table 1. In some cases, the capture probe can be a primer selected from 64F, 363F, 520F, 530F, 806R, 1027R, and 1100R. In some cases, the capture probe can be APT₂ or APT₂WO.

Provided herein are methods of making a POC diagnostic device, comprising the steps of: (a) treating a fibrous carrier with one or more reagents to functionalize fibers of the fibrous carrier with a nucleic acid binding moiety; (b) binding a capture probe to one or more of the functionalized fibers in a first discrete location of the fibrous carrier, the nucleic acid binding moiety binding the capture probe to the one or more functionalized fibers; and (c) applying a control to a second discrete location of the fibrous carrier. In some cases, the nucleic acid binding moiety is an active ester, thiocarbamate, or isothiocyanate.

In some cases, the nucleic acid binding moiety is an active thiocarbamate formed by reacting the fibrous carrier with polyamidoamine (PAMAM) dendrimer and p-phenylene diisothiocyanate (PDITC). In some cases, the nucleic acid binding moiety is an active ester formed by reacting the fibrous carrier with glutaric anhydride, N-hydroxysuccinimide, N, N′-dicyclohexylcarbodiimide (GND), or glutaric anhydride, N-hydroxysuccinimide, N, N′-dicyclohexylcarbodiimide and methanol (GND-methanol). In some cases, the one or more reagents comprises GND; 1% PAM followed by GND; 3% PAM followed by GND; 10% PAM followed by GND; GND followed by 1% PAM-GND; GND followed by 3% PAM followed by GND; GND followed by 10% PAM followed by GND; GND followed by methanol followed by GND; APTS followed by GND; APTS followed by GND followed by APTS followed by GND; APTS followed by PDITC; APTS followed by PDITC followed by APTS followed by PDITC; APTS followed by PDITC followed by APTS followed by GND; PAMAM followed by GND; PAMAM followed by GND followed by PAMAM followed by GND; PAMAM followed by PDITC; PAMAM followed by PDITC followed by PAMAM followed by PDITC; PAMAM followed by PDITC followed by PAMAM followed by GND; methanol followed by GND; or methanol followed by GND followed by methanol followed by GND.

In some cases, the fibrous carrier can be functionalized with one or more of GND, PAM, methanol, APTS, PDITC, PAMAM. For example, the filter paper (also referred to as “FP” herein) can be functionalized with 1% PAM and GND. Such a functionalized fibrous carrier is referenced herein by the nomenclature FP-1% PAM-GND. Other examples of functionalized fibrous carriers include, for example, FP-GND; FP-1% PAM-GND; FP-3% PAM-GND; FP-10% PAM-GND; FP-GND-1% PAM-GND; FP-GND-3% PAM-GND; FP-GND-10% PAM-GND; FP-GND-methanol-GND; FP-APTS-GND; FP-APTS-GND-APTS-GND; FP-APTS-PDITC; FP-APTS-PDITC-APTS-PDITC; FP-APTS-PDITC-APTS-GND; FP-PAMAM-GND; FP-PAMAM-GND-PAMAM-GND; FP-PAMAM-PDITC; FP-PAMAM-PDITC-PAMAM-PDITC; FP-PAMAM-PDITC-PAMAM-GND; FP-methanol-GND; and FP-methanol-GND-methanol-GND, and combinations thereof. In some cases, the fibrous carrier can be FP-GND. In some cases, the fibrous carrier can be FP-1% PAM-GND. In some cases, the fibrous carrier can be FP-3% PAM-GND. In some cases, the fibrous carrier can be FP-10% PAM-GND. In some cases, the fibrous carrier can be FP-GND-1% PAM-GND. In some cases, the fibrous carrier can be FP-GND-3% PAM-GND. In some cases, the fibrous carrier can be FP-GND-10% PAM-GND. In some cases, the fibrous carrier can be FP-GND-methanol-GND. In some cases, the fibrous carrier can be FP-APTS-GND. In some cases, the fibrous carrier can be FP-APTS-GND-APTS-GND. In some cases, the fibrous carrier can be FP-APTS-PDITC. In some cases, the fibrous carrier can be FP-APTS-PDITC-APTS-PDITC. In some cases, the fibrous carrier can be FP-APTS-PDITC-APTS-GND. In some cases, the fibrous carrier can be FP-PAMAM-GND. In some cases, the fibrous carrier can be FP-PAMAM-GND-PAMAM-GND. In some cases, the fibrous carrier can be FP-PAMAM-PDITC. In some cases, the fibrous carrier can be FP-PAMAM-PDITC-PAMAM-PDITC. In some cases, the fibrous carrier can be FP-PAMAM-PDITC-PAMAM-GND. In some cases, the fibrous carrier can be FP-methanol-GND. In some cases, the fibrous carrier can be FP-methanol-GND-methanol-GND.

Provided herein are POC diagnostic devices configured to display one or more visual outputs indicating the presence of a pathogen in a sample. In some cases, the devices can be configured to display one or more visual outputs indicating the presence of a bacterium, a virus, a fungus, or combinations thereof in a sample. In some cases, the devices can be configured to display one or more visual outputs indicating the presence of a bacterium in a sample. In some cases, the devices can be configured to display one or more visual outputs indicating the presence of Staphylococcus aureus, Escherichia coli, or Campylobacter jejuni. In some cases, the devices can be configured to display one or more visual outputs indicating the presence of Staphylococcus aureus. In some cases, the devices can be configured to display one or more visual outputs indicating the presence of Escherichia coll. In some cases, the devices can be configured to display one or more visual outputs indicating the presence of Campylobacter jejuni. In some cases, the devices can be configured to display one or more visual outputs indicating the presence of a fungus in a sample. In some cases, the devices can be configured to display one or more visual outputs indicating the presence of Aspergillus and/or Candida. In some cases, the devices can be configured to display one or more visual outputs indicating the presence of a virus in a sample. In some cases, the devices can be configured to display one or more visual outputs indicating the presence of SARS-CoV-2, influenza viruses, and/or Cytomegalovirus, and the like.

In accordance with the principles herein a diagnostic kit can be configured to generate an output displaying cross-reactivity of a pathogen biomarker and/or to analyze and/or confirm presence of a universal and/or specific pathogen within a fluid sample, the fluid sample identified/detected/confirmed by at least one of FP-GND; FP-1% PAM-GND; FP-3% PAM-GND; FP-10% PAM-GND; FP-GND-1% PAM-GND; FP-GND-3% PAM-GND; FP-GND-10% PAM-GND; FP-GND-methanol-GND; FP-APTS-GND; FP-APTS-GND-APTS-GND; FP-APTS-PDITC; FP-APTS-PDITC-APTS-PDITC; FP-APTS-PDITC-APTS-GND; FP-PAMAM-GND; FP-PAMAM-GND-PAMAM-GND; FP-PAMAM-PDITC; FP-PAMAM-PDITC-PAMAM-PDITC; FP-PAMAM-PDITC-PAMAM-GND; FP-methanol-GND; FP-methanol-GND-methanol-GND.

In certain embodiments the diagnostic kit can be configured such that the fluid sample can be analyzable and/or transportable via a point of care device disclosed herein. In other embodiments a test strip can be configured to generate a visual output in the presence of a pathogen or pathogens, based on detection of universal biomarkers of genes and/or proteins and selection of universal pairs of primers without cross-reactivity with human genomic DNA and/or a specific pathogen based on detection of specific markers of genes and/or proteins and selection of specific pairs of primers without cross-reactivity with human genomic DNA identified/detected/confirmed by at least one of FP-GND; FP-1% PAM-GND; FP-3% PAM-GND; FP-10% PAM-GND; FP-GND-1% PAM-GND; FP-GND-3% PAM-GND; FP-GND-10% PAM-GND; FP-GND-methanol-GND; FP-APTS-GND; FP-APTS-GND-APTS-GND; FP-APTS-PDITC; FP-APTS-PDITC-APTS-PDITC; FP-APTS-PDITC-APTS-GND; FP-PAMAM-GND; FP-PAMAM-GND-PAMAM-GND; FP-PAMAM-PDITC; FP-PAMAM-PDITC-PAMAM-PDITC; FP-PAMAM-PDITC-PAMAM-GND; FP-methanol-GND; FP-methanol-GND-methanol-GND.

Further, a universal or specific pathogen biomarker for genes and/or proteins can be identifiable from fluid samples identified/detected/confirmed by at least one of FP-GND; FP-1% PAM-GND; FP-3% PAM-GND; FP-10% PAM-GND; FP-GND-1% PAM-GND; FP-GND-3% PAM-GND; FP-GND-10% PAM-GND; FP-GND-methanol-GND; FP-APTS-GND; FP-APTS-GND-APTS-GND; FP-APTS-PDITC; FP-APTS-PDITC-APTS-PDITC; FP-APTS-PDITC-APTS-GND; FP-PAMAM-GND; FP-PAMAM-GND-PAMAM-GND; FP-PAMAM-PDITC; FP-PAMAM-PDITC-PAMAM-PDITC; FP-PAMAM-PDITC-PAMAM-GND; FP-methanol-GND; FP-methanol-GND-methanol-GND.

Additionally, a rapid process for identifying pathogens and generated samples of genes and proteins in a fluid sample can include a testing media functionalized by at least one of FP-GND; FP-1% PAM-GND; FP-3% PAM-GND; FP-10% PAM-GND; FP-GND-1% PAM-GND; FP-GND-3% PAM-GND; FP-GND-10% PAM-GND; FP-GND-methanol-GND; FP-APTS-GND; FP-APTS-GND-APTS-GND; FP-APTS-PDITC; FP-APTS-PDITC-APTS-PDITC; FP-APTS-PDITC-APTS-GND; FP-PAMAM-GND; FP-PAMAM-GND-PAMAM-GND; FP-PAMAM-PDITC; FP-PAMAM-PDITC-PAMAM-PDITC; FP-PAMAM-PDITC-PAMAM-GND; FP-methanol-GND; FP-methanol-GND-methanol-GND.

In accordance with the principles of the present disclosure a method of targeting the 16S rRNA gene can include the steps of: optimizing a selection of PCR primers to reduce cross-reaction with human DNA, thereby increasing specificity and sensitivity for BSI and other pathogen diagnosis; and configuring a capture probe using a selected PCR primer on a selected media to receive a fluid sample from a fluid source at the capture probe.

The method can further include the step of adding a reactive component to the selected PCR primer, wherein 16S rRNA gene present in the fluid sample reacts with the reactive component on or within the selected media to generate a reactive output, such as a visual output, thereby providing rapid and accurate pathogen detection for rare bacterial DNA in blood or other fluids, even in the presence of abundant host DNA.

The method can include one or more of identifying/detecting/confirming the capture probe with at least one of FP-GND; FP-1% PAM-GND; FP-3% PAM-GND; FP-10% PAM-GND; FP-GND-1% PAM-GND; FP-GND-3% PAM-GND; FP-GND-10% PAM-GND; FP-GND-methanol-GND; FP-APTS-GND; FP-APTS-GND-APTS-GND; FP-APTS-PDITC; FP-APTS-PDITC-APTS-PDITC; FP-APTS-PDITC-APTS-GND; FP-PAMAM-GND; FP-PAMAM-GND-PAMAM-GND; FP-PAMAM-PDITC; FP-PAMAM-PDITC-PAMAM-PDITC; FP-PAMAM-PDITC-PAMAM-GND; FP-methanol-GND; FP-methanol-GND-methanol-GND.

Another method in accordance with the principles herein can include the step of targeting the 16S rRNA gene by optimizing the selection of PCR primers, wherein the selection of the PCR primers increases specificity and sensitivity for pathogen detection, priorly using at least one of FP-GND; FP-1% PAM-GND; FP-3% PAM-GND; FP-10% PAM-GND; FP-GND-1% PAM-GND; FP-GND-3% PAM-GND; FP-GND-10% PAM-GND; FP-GND-methanol-GND; FP-APTS-GND; FP-APTS-GND-APTS-GND; FP-APTS-PDITC; FP-APTS-PDITC-APTS-PDITC; FP-APTS-PDITC-APTS-GND; FP-PAMAM-GND; FP-PAMAM-GND-PAMAM-GND; FP-PAMAM-PDITC; FP-PAMAM-PDITC-PAMAM-PDITC; FP-PAMAM-PDITC-PAMAM-GND; FP-methanol-GND; FP-methanol-GND-methanol-GND; and configuring the capture probe to provide reactive regions for each of the selected PCR primers in the presence of a fluid sample.

Provided herein are POC diagnostic devices configured to display one or more visual outputs indicating the presence of a pathogen in a sample. In some cases, the visual output can be labeling a bound capture probe with a visually-observable label. In some cases, the visually-observable label can be a dye, a magnetic bead, or a nanoparticle. In some cases, the visually-observable label can be a magnetic bead.

A system can be configured to produce a rapid, clear, regular and visible output signal that is observable via the naked eye, and analyzable quantitatively to indicate bacterial detection on a capture probe, such as by using 16S rDNA probes on an activated paper surface as the capture probe for universal bacterial diagnosis for example.

Systems herein can be configured to embed a selection of PCR primers on a capture probe, and further configured to generate an output indicating presence of the 16S rRNA gene, wherein the system can be optimized to avoid cross-reaction with human or animal DNA background in samples derived from or containing human or animal fluid.

If desired, the system can provide rapid and accurate pathogen detection for rare bacterial DNA in blood in the presence of abundant host DNA while also increasing the specificity and sensitivity for BSI diagnosis.

In certain embodiments a Point-of-care (POC) detection device can include: polyamidoamine (PAMAM) dendrimer, and p-Phenylene diisothiocyanate (PDITC), configured on a filter paper in order to activate the surface of filter paper to bind DNA molecules from a sample containing DNA.

The POC device can be formed by a process including the steps of primary amination of the surface of filter paper with PAMAM dendrimer, followed by creating isothiocyanate groups via PDITC, and subsequently repeating these two steps.

The POC device can be further defined by a filter paper formed of a highly porous structure, such that multiple printed probes, target DNAs and indicators, such as magnetic beads, can be embedded therein and provide high signal intensities in the detection area via probe/target duplex formation.

The POC device can be configured to carry out a rapid, specific and cost-efficient DNA detection, wherein the filter paper is further defined by cellulose filter paper.

The POC device can further include embedded treatment materials, such that the device can be configured to connect a sample to the capture probe(s) for diagnosis and to selectively release treatment for an infectious disease, where the device can be configured to further facilitate identification of antimicrobial drug resistance genes based on a small sample size.

A point of care device (POC) can include: surface-functionalization systems of cellulose filter paper including glutaric anhydride, N-hydroxysuccinimide, N, N′-Dicyclohexylcarbodiimide and methanol.

In some embodiments the POC device can be configured to identify synthetic oligonucleotides and bacterial genomic DNA.

In certain embodiments a filter paper can include an indicator, such as superparamagnetic beads, configured to selectively bind to a capture probe in the presence of a sample containing universal or specific pathogen and to display a visual signal when so bound due to an iron color produced by the superparamagnetic beads.

The filter paper can include capture probes configured to be bound with the superparamagnetic beads, wherein the superparamagnetic beads are collectable and purifiable via a magnetic stand.

Also provided herein are methods for detecting the presence of a pathogen in a sample, the methods comprising: (a) contacting the sample comprising or suspected of comprising a target nucleic acid sequence from the pathogen with a visual label under conditions to bind the visual label to a target nucleic acid sequence and/or target protein thereby providing a labeled sample; (b) contacting a POC diagnostic device described herein with the labeled sample, wherein upon contact the target nucleic acid sequence if present binds to one or more of the capture probes congregating the visual label attached to the nucleic acid sequence pathogen in the first discrete location and generating a visual output; and (c) washing the device to remove unbound portions of the sample. In some cases, the pathogen is a bacterium, a virus, or a fungus. In some cases, the bacterium can be Staphylococcus aureus, Escherichia coli, or Campylobacter jejuni. In some cases, the bacterium can be Staphylococcus aureus. In some cases, the bacterium can be Escherichia coli. In some cases, the bacterium can be Campylobacter jejuni. In some cases, the pathogen can be a virus. In some cases, the virus can be SARS-CoV-2, influenza viruses, and/or Cytomegalovirus. In some cases, the pathogen can be a fungus. In some cases, the fungus can be Aspergillus and/or Candida.

In some cases, the methods of the disclosure can include contacting the device with the sample without any prior treatment of the sample. In such cases, circulating DNA and/or RNA from the target pathogen present in or suspected to be present in the sample can be detected. In some cases, pre-treatment of the as-taken sample can be done to release, extract and/or concentrate DNA and/or RNA from the target pathogen prior to detecting using the devices of the disclosure. In some cases, the pre-treatment comprises extraction of a target nucleic acid from the sample, amplification by PCR, and/or denaturation of double stranded DNA. In some cases, the pre-treatment comprises extraction of target DNA and/or target RNA from the sample, amplification by PCR, and/or denaturation of double stranded DNA. pre-treatment of the sample can include exposing the sample to conditions sufficient to lyse cells present in the sample to release their DNA and/or RNA. Any combination of pretreatment steps can be done. Other methods of pre-treatment to release and/or concentrate and/or amplify DNA and/or RNA in a sample are known to those skilled in the art, e.g., chemical and electrical means.

In some cases, the sample can be a fluid sample. In some cases, the sample can be a bodily fluid. In some cases, the sample can be blood, urine, saliva, breast milk, mucus, pus, sweat, tears, cerebrospinal fluid (CSF), semen, serum, plasma, or bronchoalveolar lavage fluid, or combinations thereof. In some cases, the sample can be blood. In some cases, the sample can be fluid used in the manufacturing of pharmaceutical or food products. In some cases, the sample can be water. In some cases, the sample can be metalworking fluid, coolant, or potable water. In some cases, the sample can be potable water.

In some cases, the volume of the sample can be about 1 μL to about 100 mL. In some cases, the volume of the sample can be about 0.01 mL to about 10 mL. In some cases, the volume of the sample can be about 0.1 mL to about 1 mL. In some cases, the volume of the sample can be about 0.01 mL to about 1 mL. In some cases, the volume of the sample can be about 0.01 mL to about 0.5 mL. In some cases, the volume of the sample can be about 0.01 mL. In some cases, the volume of the sample can be about 0.05 mL. In some cases, the volume of the sample can be about 0.1 mL. In some cases, the volume of the sample can be about 0.5 mL. In some cases, the volume of the sample can be about 1 mL.

In some cases, detecting the presence of a pathogen can be detecting the presence of a pathogen target. In some cases, detecting the presence of a pathogen can be detecting the presence of a nucleic acid or protein. In some cases, detecting the presence of a pathogen can be detecting the presence of a nucleic acid. In some cases, the nucleic acid can be genomic DNA or genomic RNA. In some cases, the nucleic acid can be genomic DNA. In some cases, the genomic DNA can be a 16S rRNA gene. In some cases, the genomic DNA can be an antimicrobial resistance gene. In some cases, the nucleic acid can be genomic RNA. In some cases, detecting the presence of a pathogen can avoid cross-reaction with background human or animal DNA present in the sample. In some cases, detecting the presence of a pathogen can avoid cross-reaction with background human DNA present in the sample. In some cases, the nucleic acid can be a primer as recited in Table 1, Table 2, Table 3, or Table 4. In some cases, the nucleic acid can be a primer as recited in Table 1. In some cases, the nucleic acid can be a primer as recited in Table 2. In some cases, the nucleic acid can be a primer as recited in Table 3. In some cases, the nucleic acid can be a primer as recited in Table 4.

In some cases, detecting the presence of a pathogen can comprise detecting an amplicon originating from PCR amplification of a sample. In some cases, the amplicon is a DNA amplicon. In some cases, the amplicon is an RNA amplicon.

In some cases, the visual output can indicate the quantity or identity of a pathogen in a sample. In some cases, the visual output can indicate the quantity of a pathogen in a sample. In some cases, the visual output can indicate the identity of a pathogen in a sample. In some cases, the visual output can indicate the quantity and identity of a pathogen in a sample. In some cases, the visual output can indicate the quantity, identity, or both quantity and identity of a pathogen in a sample based on a variation in intensity of the visual output. In some cases, the visual output can indicate the quantity, identity, or both quantity and identity of a pathogen in a sample based on an increase in intensity of the visual output. In some cases, the visual output can indicate the quantity, identity, or both quantity and identity of a pathogen in a sample based on a decrease in intensity of the visual output. In some cases, the visual output results from a chemical reaction between the capture probe of the device and the sample. In some cases, the visual output results from interaction between the capture probe of the device and the sample. In some cases, the visual output results from binding between the capture probe of the device and the sample. In some cases, the visual output can be a color change. In some cases, the visual output can be superparamagnetic beads.

The quantity of pathogen in the sample can be determined using techniques described herein, e.g., the technique described in Example 2. Identity of the pathogen in the sample can be determined using techniques described herein, e.g., by printing multiple capture probes and indicia identifying various pathogens.

In some cases, the superparamagnetic beads selectively bind to a pathogen target. In some cases, the superparamagnetic beads can be iron oxide nanoparticles. In some cases, the superparamagnetic beads can be gold nanoparticles (AuNPs).

In some cases, the sample can be contacted with a label that creates a visual output when a target in the sample binds to a capture probe embedded in the devices disclosed herein. In some cases, the sample can be contacted with the label prior to the sample being contacted with the device. In some cases, the sample can be contacted with the label after being contacted with the device.

In some cases, the sample can be contacted with the label for a defined period of time. In some cases, the sample can be contacted with the label for about 1 minute to about 20 minutes. In some cases, the sample can be contacted with the label for about 1 minute, about 2 minutes, about 3 minutes, about 4 minutes, about 5 minutes, about 6 minutes, about 7 minutes, about 8 minutes, about 10 minutes, about 11 minutes, about 12 minutes, about 13 minutes, about 14 minutes, about 15 minutes, about 16 minutes, about 17 minutes, about 18 minutes, about 19 minutes, or about 20 minutes. In some cases, the sample can be contacted with the label for about 1 minute. In some cases, the sample can be contacted with the label for about 5 minutes. In some cases, the sample can be contacted with the label for about 10 minutes. In some cases, the sample can be contacted with the label for about 15 minutes. In some cases, the sample can be contacted with the label for about 20 minutes.

In some cases, the sample can be contacted with the probe for a defined period of time. In some cases, the sample can be contacted with the probe for about 1 minute to about 20 minutes. In some cases, the sample can be contacted with the probe for about 1 minute, about 2 minutes, about 3 minutes, about 4 minutes, about 5 minutes, about 6 minutes, about 7 minutes, about 8 minutes, about 10 minutes, about 11 minutes, about 12 minutes, about 13 minutes, about 14 minutes, about 15 minutes, about 16 minutes, about 17 minutes, about 18 minutes, about 19 minutes, or about 20 minutes. In some cases, the sample can be contacted with the probe for about 1 minute. In some cases, the sample can be contacted with the probe for about 5 minutes. In some cases, the sample can be contacted with the probe for about 10 minutes. In some cases, the sample can be contacted with the probe for about 15 minutes. In some cases, the sample can be contacted with the probe for about 20 minutes.

### Example 1: Identification of 16S rRNA Primers with No Similarity to the Human Genome

Bloodstream infection (BSI) is a severe medical condition associated with increased morbidity and mortality worldwide, with and incidence of 80-200 per 100,000 annually. The routine detection method of BSI is blood culture, and the most commonly detected bacterial strains are Escherichia coli, Staphylococcus aureus and Streptococcus pneumoniae. Blood culture can only detect culturable pathogens, and it takes several days to perform. Molecular methods can also be used for diagnosing BSI: they can be executed in a few hours, and can detect a wide variety of bacteria by targeting multiple strains using specific probes or universal marker genes.

The 16S rRNA gene is a ubiquitous gene present in all bacteria, and therefore is often used for universal bacterial detection in BSI. However, there is a large amount of host DNA present in blood samples, which hampers the sensitivity and specificity of molecular BSI diagnosis, due to the similarity between bacterial and human genomes. In accordance with the principles herein primers were selected which efficiently amplify the 16S rRNA gene, but do not cross-react with the human genome.

Unlike current approaches to avoid or reduce human DNA background in 16S detection of blood samples, which include the use of less PCR cycles what may result in reduced sensitivity, or enzymatic approaches, which may increase workload and cost, a significant improvement in the sensitivity and specificity of bacterial DNA detection in BSI is achievable by avoiding cross-reactivity between PCR primers targeting the 16S rRNA gene and the host background DNA in accordance with the principles herein.

All available primer sequences (n=113) targeting the conserved segments of the 16S rRNA gene can be downloaded from ProbeBase (Table 1). Similarity search to the human genome and its transcripts can be performed using BLAST. In addition, sequences can be mapped to the human genome (GRCh v38) using the CLC Genomic Workbench. Primer pairs can be selected for further testing based on 1) their melting temperatures 10° C. difference between forward and reverse primers) and 2) their sizes in order to identify primer pairs for high throughput sequencing (550 base pair) and Sanger sequencing (700 base pair). Melting temperatures of primers can be determined using OligoCalc.

Cross-Reactivity of Primers with Human Genome

The specificity and sensitivity of the selected primers can be tested in a PCR reaction with E. coli and human genome (Sigma) as templates. PCR conditions can include the following: 200 nM primers, lx Kapa PCR mix (Kapa Biosystems), 3 μl template in a 20 μl reaction, or other suitable conditions. Tubes can be incubated in a thermocycler at 95° C. for 3 min, then cycled at 95° C. for 30 sec, 50° C. for 30 sec, and 72° C. for 30 sec for 25 times, for example.

Sensitivity of the primer pairs can be tested with a mixture of human and E. coli genomes, by modeling conditions of BSI. Standard amount of human genome (11 ng/μl) and decimal dilutions of E. coli (55 ng/μl to 55 fg/μl) can be used as templates. The 341F and 803R primers can be added as reference as they are used for sequencing studies.

### Sequencing

Two-hundred μl PCR amplicons, or other suitable amount can be used for Sanger sequencing to identify the amplified products using an AB 3730xl instrument, for example. Sequences can be analyzed using BioEdit 7.2.5 software or other suitable software.

Sequence Similarity of Primers with the Human Genome

One hundred-thirteen primers can be analyzed, and 7 primers (6.2%) can be identified with 0% similarity (64F, 363F, 520F, 530F, 806R, 1027R, 1100R, Table 2) to the human genome and its transcripts.

Cross-Reactivity of Primers with the Human Genome

Blood samples from BSI contain a large amount of human DNA background that can result in false positive (cross-reactivity with the human genome) or false negative (due to reduced sensitivity) detection of bacteria. Therefore, the specificity of selected primer pairs using E. coli and human genomic DNA can provide cross-reactivity information.

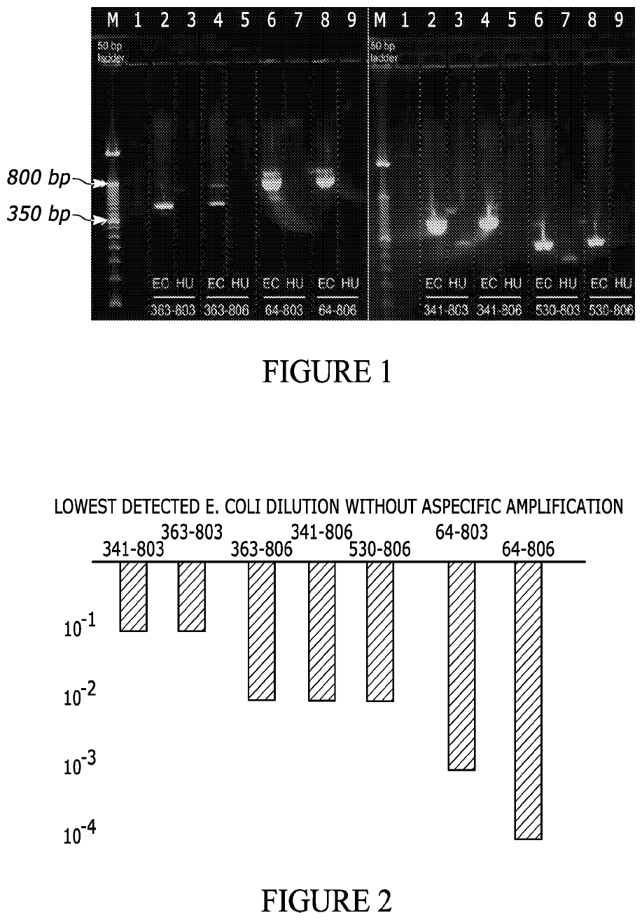

For example, the reference primer 341F resulted in false amplification with all other primers, similarly to the 1027R primer, as it also generated false amplification in all combinations (Table 3, FIG. 1). Apart from their size, the amplicons were also identified by Sanger sequencing, and their origins (human or bacterial) were confirmed in all cases. The following combinations specifically amplified E. coli but not the human genome: 64-803, 64-806, 363-803, 363-806, 530-806 (Table 3, FIG. 1).

Sensitivity of PCRs with Different Primer Pairs with Human Background DNA

Samples from BSI always contain a large amount of human DNA, which can result in misamplification, false positive results and decreased sensitivity of PCR systems. In order to model BSI, human DNA (in a standard amount) was mixed with serial dilutions of E. coli genomic DNA. Amplicons were subjected to Sanger sequencing and identified by BLAST. Sensitivity was calculated based on the resulted E. coli amplicons. Only the reference primer pair and those primers which did not cross-react with the human genome were tested.

Rapid, sensitive and specific detection of pathogens is crucial in BSI cases. Antimicrobial treatment has to be started within hours after symptoms of BSI develops, because delay in antimicrobial treatment is associated with increased mortality in BSI. Accurate identification of pathogens is important, as false positive results would promote antibiotic misuse, generation of antimicrobial resistance and dysbiosis of the indigenous microbiota.

The known routine BSI diagnostic method (blood culture) has limited detection range, requires extensive instrumentation and takes days to obtain results. Rapid molecular methods are available for BSI diagnosis, and can target universal bacterial marker genes to allow the detection of unculturable pathogens.

However, contrary to most metagenomics samples (e.g., feces), blood contains a large amount of host DNA and low levels of bacterial DNA, which can decrease the specificity of molecular diagnostic systems due to cross-reactivity. Sensitivity is also crucial in BSI diagnosis, as pathogen concentration can be as low as 1 CFU/ml, and cross-reaction of primers results in less sensitive detection. To address this problem, a bacterial detection system in accordance with the principles herein was optimized based on the amplification of the 16S rRNA gene, which show improved specificity (FIG. 1) and sensitivity (FIG. 2) by avoiding cross-reactivity with human genome.

Thus primer pairs for BSI detection for high-throughput sequencing systems (341-806, 363-806) and for traditional Sanger sequencing (64-803 or 64-806) can be achieved without amplifying human DNA. As a consequence of the reduction of human DNA background amplification, PCR efficiency can be significantly improved by this approach (FIG. 2).

In cases where the sizes of amplicons of human and bacterial samples are different (e.g., the 341-803 primer), size selection may be applicable to eliminate or decrease human background (FIG. 1, 341-803 or 530-803 primer pairs), but it is a laborious process, with risk of PCR contamination. In accordance with the principles herein amplicons with optimized 16S rRNA primers can be used without further size selection as they do not result in misamplification.

In summary, devices and methods in accordance with the principles herein provide a sensitive and specific amplification of the 16S rRNA gene with appropriate selection of primers for BSI, or for other samples where a significant amount of human background DNA is present, or for still other samples where portable detection of pathogens is desired as discussed herein. These results can be utilized for molecular diagnosis of BSI, including high-throughput sequencing and PCR-based approaches, for example.

TABLE 1

| | | SEQ | Tm |
|---|---|---|---|
| Probebase | | ID | (Oligo |
| Name | Sequence 5′-3′ | NO: | Calc) |
| 806R | GGA CTA CHV GGG TAT CTA | 1 | 47.7-51.8 |
| 1027R | CGA CRR CCA TGC ANC ACC T | 2 | 51.1-57.6 |
| 1100R | GGG TTN CGN TCG TTG | 3 | 41.9-47.4 |
| 520F | AYT GGG YDT AAA GNG | 4 | 33.7-44.7 |
| 530F | GTG CCA GCM GCN GCG G | 5 | 53.6-58.8 |
| 64F | BGY CTW ANR CAT GCA AGT | 6 | 45.6-55.9 |
| 363F | CAA TGG RSG VRA SYC TGA | 7 | 49.7-60 |
| Bakt805R | GAC TAC HVG GGT ATC TAA | 8 | 50.5-54.4 |
| U751F | CCG ACG GTG AGR GRY GAA | 9 | 50.3-57.2 |
| 986F | CNA CGC GAA GAA CCT TAN C | 10 | 48.9-53.2 |
| 6F | ATT CYG GTT GAT CCY GSC | 11 | 51.8-57.9 |
| P1425 | WAG GAG GTR ATC CAD CC | 12 | 44.6-49.5 |
| 802R | TAC NVG GGT ATC TAA TCC | 13 | 43.5-48 |
| P609D | GGM TTA GAT ACC CBD GTA | 14 | 43.5-50.3 |
| 917Fw | GAA TTG ACG GGG RCC CGC A | 15 | 55.4-57.6 |
| Bac967Fd | ATA CGC GAR GAA CCT TAC C | 16 | 48.9-51.1 |
| 803R | CTA CCR GGG TAT CTA ATC C | 17 | 48.9-51.1 |
| Bac1046Rd | CGA CGA CCA TGC ANC ACC T | 18 | 53.2-55.4 |
| 907R | CCG TCA ATT CMT TTG AGT TT | 19 | 45.6-47.7 |
| 68F | TNA NAC ATG CAA GTC GRR | 20 | 47.7-55.9 |
| P609R | TAC HVG GGT ATC TAA KCC | 21 | 43.5-50.3 |
| 1061Rv | TCA CGR CAC GAG CTG ACG | 22 | 55.9-57.9 |
| 1099F | GYA ACG AGC GCA ACC C | 23 | 48.5-51.1 |
| 1114R | GGG TTG CGC TCG TTR C | 24 | 48.5-51.1 |
| Ab789F | TAG ATA CCC SSG TAG TCC | 25 | 50.3 |
| Bact531R | CTN YGT MTT ACC GCG GCT | 26 | 53.8-60 |
| 1061R | CRR CAC GAG CTG ACG AC | 27 | 49.5-54.3 |
| U341F | CCT ACG GGR SGC AGC AG | 28 | 54.3-56.7 |
| Uni522R | GWA TTA CCG CGG CKG CTG | 29 | 52.6-54.9 |
| Bac1046Rb | CGA CAA CCA TGC ANC ACC T | 30 | 51.1-53.2 |
| Bac967Fe | CTA ACC GAN GAA CCT YAC C | 31 | 48.9-53.2 |
| Uni1392R | ACG GGC GGT GTG TRC | 32 | 47.4-50.1 |
| 1050R | ACG ACA GCC ATG CAN C | 33 | 45.9-48.5 |
| 1407R | GAC GGG CGG TGT GTR C | 34 | 51.1-53.6 |
| 970F | CGC GAA GAA CCT TAC C | 35 | 45.9 |
| U1053F | GCA TGG CYG YCG TCA G | 36 | 48.5-53.6 |
| U1053R | CTG ACG RCR GCC ATG C | 37 | 48.5-53.6 |
| Bact806R | GGA CTA CCA GGG TAT CTA | 38 | 58 |
| 908R | CGT CAA TTC MTT TGA GTT | 39 | 41.2-43.5 |
| 536R | CAG CMG CCG CGG TAA TWC | 40 | 52.6-54.9 |
| 909F | ACT CAA AKG AAT WGA CGG | 41 | 43.5-45.8 |

TABLE 2

| | Sequence 5′-3′ |
|---|---|
| 363F | CAA TGG RSG VRA SYC TGA HS SEQ ID NO: 42 |
| 64F | BGY CTW ANR CAT GCA AGT YG SEQ ID NO: 43 |
| 520F | AYT GGG YDT AAA GNG SEQ ID NO: 44 |
| 530F | GTG CCA GCM GCN GCG G SEQ ID NO: 45 |
| 806R | GGA CTA CHV GGG TAT CTA AT SEQ ID NO: 46 |
| 1027R | CG ACRR CCA TGC ANC ACC T SEQ ID NO: 47 |
| 1100R | GGG TTN CGN TCG TTG SEQ ID NO: 48 |

TABLE 3

| | Reverse | | |
|---|---|---|---|
| Forward | 803 | 806 | 1027 |
| 64 | +/− | +/− | +/+ |
| 341 | +/+ | +/+ | +/+ |
| 363 | +/− | +/− | +/+ |
| 530 | +/+ | +/− | +/+ |

Point-of-care (POC) detection is crucial in clinical diagnosis in order to provide timely and specific treatment. Combining polyamidoamine (PAMAM) dendrimer, p-Phenylene diisothiocyanate (PDITC) and superparamagnetic beads, methods to activate the surface of filter paper to bind DNA molecules can be achieved. Methods herein are based on the steps of primary amination of the surface of filter paper with PAMAM dendrimer, followed by creating isothiocyanate groups via PDITC, and subsequently repeating these two steps. Different parameters of the processes can be optimized including probe printing, preparation of target DNAs and detection.

The result shown in the figures indicates that, due to the highly porous structure of filter paper, high amount of printed probes, target DNAs and magnetic beads can provide high signal intensities in the detection area via probe/target duplex formation. Thus, methods in accordance with the principles herein are suitable for a rapid, specific and cost-efficient DNA detection on cellulose filter paper, or other suitable carrier. The technique can be used as a POC device, if desired, in particular for diagnosis and treatment management of infectious diseases, identification of antimicrobial drug resistance genes, or rapid pathogen detection in any fluid sample.

Filter paper (FP) or other suitable carrier has considerable advantages over traditional DNA hybridization platforms regarding turnaround time, price and ease of use. Since FP possesses three-dimensional microstructure, and its pore size is larger than that of nitrocellulose, FP provides a stronger wicking force and higher surface-to-volume ratio for fluid to transport. Therefore, FP is a suitable material for low-cost and easy-to-use POC devices for health care.

### Example 2: Immobilization and Detection of DNA Using Filter Paper

In accordance with the principles herein, methods and devices configured to activate the surface of FP to bind DNA by combining principles of LFAs and traditional microarray, makes a rapid, specific, instrument-free and cost-efficient detection possible. Thus, devices and methods in accordance with the principles herein provide a simplified diagnostic platform, which can be applied to bedside diagnosis of rapidly progressive diseases.

To this end, Polyamidoamine (PAMAM) dendrimer (such as 10% in methanol), p-Phenylene diisothiocyanate (PDITC), Dimethyl sulfoxide (DMSO), WHATMAN™ Qualitative filter paper, and 3-aminopropyltriethoxysilane (APTS), glutaric anhydride (GA), N-hydroxysuccinimide (NHS), N,N′-Dicyclohexylcarbodiimide (DCC), N,N-dimethylformamide (DMF) and the like are examples of suitable materials in accordance with the principles herein. PAMAM dendrimer in aqueous solution at 10% solids is yet another material. DYNAL MyOne Dynabeads Streptavidin Cl are an example of a suitable indicator material. A magnetic stand, such as MagRach16, Germany can serve as a system component in accordance with the principles herein. Suitable examples of DNA Oligonucleotides are listed in Table 4.

### Effect Comparison of Methanol and PAMAM Dendrimer to Activate the Surface of FP

Although a wide variety of materials and sizes can serve as a carrier material, in accordance with the principles herein, the discussion below is based on activating 10-12 mm×15 mm paper slides. Eight different methods were tested and compared (Table 5).

Method (1) FP-GND: first, the FP can be soaked into the saturated GA in DMF overnight to produce carboxylic groups on the surface of FP; second, to prepare the active ester compounds containing carboxylate via reacting with 1M of NHS and 1M of DCC in DMF for 4 hours, for example. These active ester compounds can be conjugated with amines via amide bonds.

Methods (2-4) 1%/3%/10% PAM-GND: FP surface can be aminated with PAMAM dendrimer in methanol for 24 hours, followed by the production of carboxylic ester groups described in method (1).

Methods (5-7) GND-1%/3%/10% PAM-GND: The carboxylic ester groups can be generated first via the reactions between the saturated GA and 1M of NHS/DCC in DMF on FP surface, followed by the process of methods (2-4).

Method (8) GND-methanol-GND: Methanol can be used to take the place of PAMAM in methods (5-7), for example. Untreated FP can be used as a negative control. All the above steps can be completed at room temperature.

### Further Comparison of Active Effect of APTS and PAMAM During Combinations of PDITC or GA-DCC-NHS or PDITC/GA-DCC-NHS

As shown in Table 6, APTS-GND: Amino groups (—NH₂) can be produced through the reaction between APTS (volume ratio of APTS:H₂O:ethanol=2:3:95) and hydroxyl groups (OH) of FP for 24 hours. The APTS solution can be added to the FP slides. Amine-reactive ester compounds on FP surface can be prepared by coating with saturated GA in DMF overnight followed by reaction with 1M of NHS/DCC in DMF for 4 hours at room temperature, for example.

APTS-GND-APTS-GND: the steps of APTS-GND can be repeated on the same FP. APTS-PDI: FP slides can be immersed in 10 μM of PDITC in DMSO after APTS overnight incubation to create isothiocyanate groups on the surface of FP, for example. APTS-PDI-APTS-PDI: APTS-PDI can be repeated on the same FP. APTS-PDI-APTS-GND: the last step of APTS-PDI-APTS-PDI can be modified by GND to prepare active ester groups. For example, 100 μL of PAMAM dendrimer (10% in methanol) can take the place of APTS in all the above five methods.

Thus, the following methods were also evaluated respectively: PAM-GND, PAM-GND-PAM-GND, PAM-PDI, PAM-PDI-PAM-PDI and PAM-PDI-PAM-GND. The operational guideline is to immerse the entire paper slide into the solution containing the APTS or dendrimer.

### Washing and Drying

After reactions with GA in DMF and NHS/DCC in DMF, the FP slides can be washed by DMF in both of the steps. After the steps either with PAMAM in methanol or only with methanol, methanol itself can be applied to wash the paper slides. Ethanol can be used to wash FP after amino groups from APTS are created on the surface of FP, for example. DMSO can be used in wash step after activation with isothiocyanate groups from PDITC in DMSO. All wash steps can be for 5 minutes followed by water wash for 3 minutes then FP slides can be dried, for example. The entire procedure of functionalization can be completed at room temperature.

### Probes and Target

In accordance with the principles herein, three probes were immobilized on each activated surface of FP. The structure of probes includes amino group modification, carbon 6 or 12 spacer, polythymine (15dT) spacer and main body of a sequence from 5′ end to 3′ terminal (Table 4). All the probes were aminated at 5′ end of oligonucleotides except for that of APT₂WO. The main sequences of APT₂ and APT₂WO were the same, and the ones of APT₂ and TID were from Chumphukam and Araújo et al, respectively. The complementary sequences of main body of APT₂ (RE-APT₂) was biotinylated at 5′ terminal and was used to detect the probe of aminated-APT₂ printed on the surface of activated FP.

### Probe Immobilization

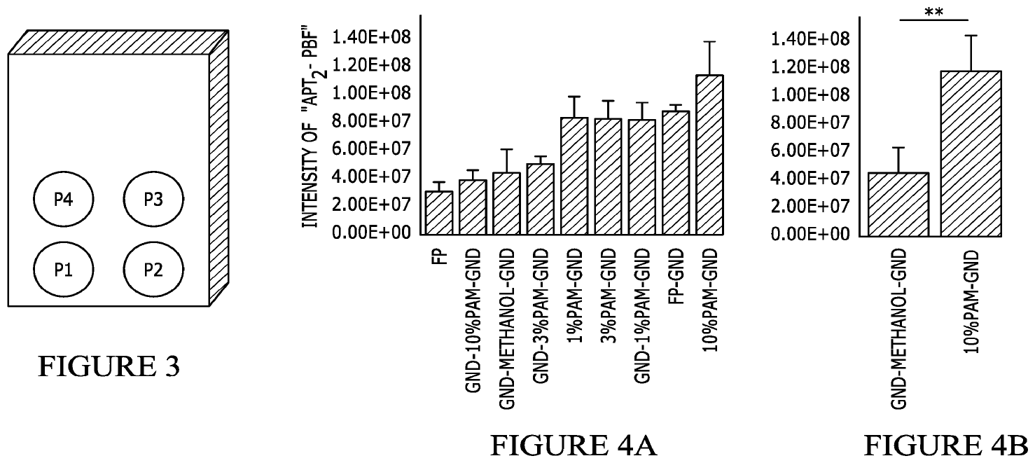

The size of each FP slide in this example was 10-12 mm×15 mm. There were four areas for probe immobilization (FIG. 3). One point five microliter of each 20 μM probe was printed in position 1 (APT₂), position 3 (TID) and position 4 (APT₂WO) manually. The probes were dissolved in 1×printing buffer (50 mM sodium phosphate buffer at pH 8-9) (PBF), therefore, PBF was printed in position 2 as a blank control. TID and APT₂WO were two negative controls. The FP slides were incubated for 24 hours in a humid chamber at room temperature. The unreacted active groups on the surface of FP were blocked with the solution of 50 mM ethanolamine and 100 mM Tris, pH 9.0 at 55° C. for 30 minutes. It was followed by the wash with 4×SSC buffer with 0.1% SDS for 30 minutes at 55° C. and then the printed FP slides were dried at room temperature.

### Preparation of Single-Stranded DNA Bound Magnetic Beads and Detection

Streptavidin-coated paramagnetic beads were used to label biotinylated single-stranded target (RE-APT₂). RE-APT₂ (e.g. 30 μmol) in 1×bind/wash buffer (0.01 M Tris-HCl, 1 mM EDTA, 2M NaCl, 1 mM 3-Mercaptoethanol, 0.1% Tween 20, pH 7.5) was incubated with paramagnetic beads (e.g. 6 μL) in a rotator for 10 minutes at room temperature. The suspension was removed after the tube was incubated on a magnetic stand for 2 minutes. FP was then washed with 2004 of 1×PBS-T twice. The single-stranded DNAs (ssDNAs) labeled with magnetic beads were dissolved in 1×PBS-T. For each paper slide detection, 50 μL of 1×PBS-T was needed. Detection was done twice on each paper slide (25 μL×2).

The printed paper slide was vertically touched into 254 of RE-APT₂ labeled magnetic beads solution in PBS-T for around 2 minutes. FP was then washed in 2×SSC buffer with 0.1% SDS at 55° C. for 10 minutes, following a wash with 0.2×SSC buffer for 1 minute at room temperature, a wash with water for 1 minute, and finally was dried at room temperature. The repeated detection was carried out with the left 254 of RE-APT₂-beads solution on the same paper slide as described above. Triplicated detections of each type of active slide were done at one time.

### Image and Statistics Analysis

Pathogen quantification using the methods and devices disclosed herein can be achieved using the technique described below.

The myImageAnalysis v1.1 software can be used to measure signal intensities, for example. The signal intensity from each printed area can be abstracted in each FP slide. The intensities of ‘APT₂-PBF’ (subtracting the volume of position 2 from that of position 1) can be analyzed and compared amongst the different methods. Student's t-test can be used to estimate significance, and p≤0.05 can be set as significant level.

### Optimization of Parameters in the Selected Method

Based on the method of PAMAM-PDITC-PAMAM-PDITC, three concentrations of PDITC (10 mM, 20 mM and 30 mM), two different volumes (80 μL and 100 μL) of PAMAM dendrimer in methanol and two kinds of carbon spacers (carbon 6 and carbon 12) can be used to optimize the method. Thirty picomoles of target ssDNAs can be bound with 64 magnetic beads, and detected 1.54 of printed probes (20 μM). The signal intensities of ‘APT₂-PBF’ can be calculated and compared under different conditions listed above, for example.

Comparison of Activation Effect on FP Surface with PAMAM Either in Methanol or in Deionized Water

The efficacy of functionalization with PAMAM dendrimer in methanol and in deionized water (pH=8.5 and pH=4.6) can be determined according to the following example. To this end, one hundred microliter of PAMAM dendrimer in methanol and 1504 of PAMAM dendrimer in deionized water can be used with 20 μM of PDITC in DMSO and APT₂ probes modified with C12 at 5 terminal end. One point five microliter of printed probe (20 μM), 34 of magnetic beads and 1.54 of target oligonucleotide (10 μM) can be applied to complete printing and detection. The signal intensities of ‘APT₂-PBF’ can be abstracted from active FP, then analyzed and compared. The activated FP slides can be aminated with 10% PAMAM in methanol and that in deionized water (wt % solids), respectively. Paper slide size can be 12 mm×15 mm.

### Optimization of Amounts of Immobilized Probes and Target Oligonucleotides and Volume of Magnetic Beads

To optimize the amount of immobilized probes, three different volumes of each probe (1.5 μL, 3 μL and 6 μL) can be tested sequentially in the same printed layout. One time PBF can be used as a blank control. Three microliter of magnetic beads and 1.54 of target oligonucleotides (10 μM) can be used for detection. In order to investigate the amount of target ssDNAs, 30 μmol and 60 μmol of target ssDNAs can be used to detect 6 μL of printed probes (20 μM) on the active FP, combining 34 of beads in each condition. Later, three distinct volumes of beads (3 μL, 4.5 μL and 6 μL) can be compared by identifying 64 of printed probes bound with C12 (20 μM) on the activated surface of FP. Sixty picomoles of target nucleic acid can be used in the detections. The signal intensities of ‘APT₂-PBF’ can be compared in all optimizations.

### Evaluation of the Efficiency of Surface-Activation-Systems on Cellulose Filter Paper

The fourth generation PAMAM dendrimer contains 64 primary amino groups (—NH2) in outer sphere. There are potentials for PAMAM to increase the surface area of cellulose filter paper based on its unique porous structure comparing with glass or nitrocellulose, so as to increase density and homogeneity of signal intensities. Thus, PAMAM in methanol (in different concentrations) can be investigated to evaluate the efficiency of surface activation systems on cellulose filter paper.

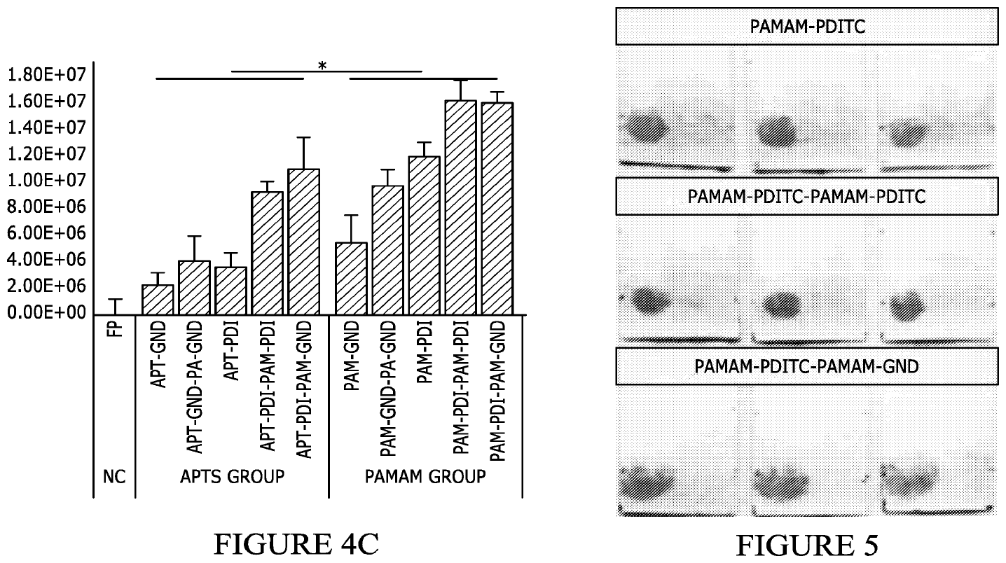

Based on GA, NHS/DCC, PAMAM dendrimer and methanol, eight different methods were investigated (FIGS. 4A and 4B) by using different concentrations of PAMAM dendrimer. There was a positive correlation between concentrations of PAMAM and signal intensities in the method of PAMAM-GND, yet it was negatively correlated with that of GND-PAMAM-GND (FIG. 4A). The method of 10% PAMAM-GND displayed the highest intensity while compared with the signals in another seven methods (FIG. 4A). The difference between 10% PAMAM-GND and GND-Methanol-GND was significant (p=0.008).

APTS is commonly used in the derivatization process of an active surface of glass slide. PDITC was homobifunctional compounds that formed polymeric network after their reactions with multiple amino groups of PAMAM. The method that GA, DCC, and NHS were applied to activate step by step avoided the crosslink formed in one step and provided a higher signal compared with the method containing PDITC.

Nine methods were investigated to evaluate the activation effect with APTS or 10% PAMAM dendrimer in methanol (wt % solid) with combinations of GA-NHS-DCC or PDITC or PDITC together with GA-NHS-DCC. The method of PAMAM-GND with 10% dendrimer was compared with these nine methods. Non-treated FP was used as negative control (FIG. 4C). The result presents the signal intensities of methods with PAMAM significantly higher than that with APTS (p=0.03). The strongest signal intensity was obtained from treatment with PAMAM-PDITC-PAMAM-PDITC (FIG. 4C and FIG. 5). There was a significant difference between the signal intensities of the methods of PAMAM-GND and PAMAM-PDITC-PAMAM-PDITC (p=0.001, FIG. 4C).

### Evaluation of Parameters in the Method of PAMAM-PDITC-PAMAM-PDITC

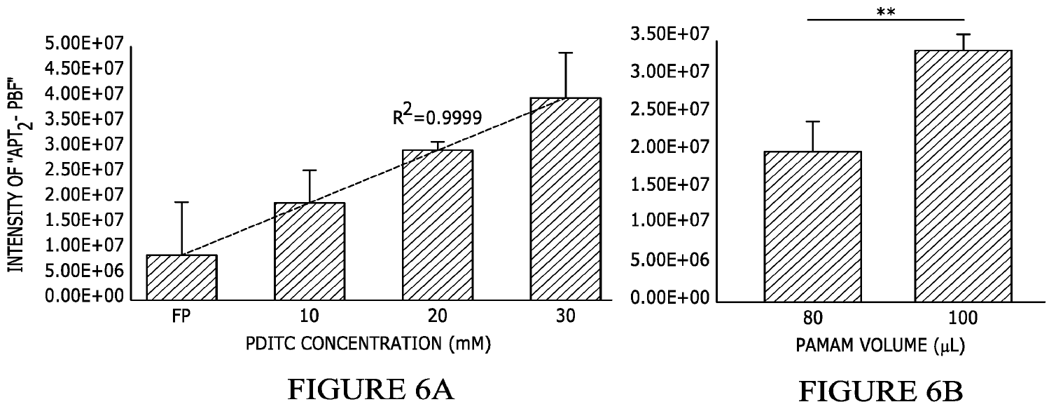

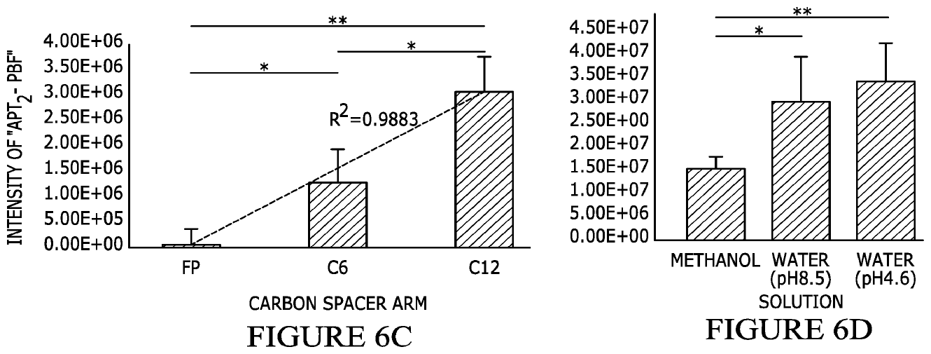

The effect of 10 mM, 20 mM and 30 mM of PDITC was investigated based on the method of PAMAM-PDITC-PAMAM-PDITC and analyzed the signal intensities by subtracting the volume of PBF from that of APT₂. The strongest signal was detected when using 30 mM of PDITC and the increase in concentration resulted in a linear increase in signal intensity (FIG. 6A, R²=0.99). Significant difference was presented between the signal intensities of 10 mM and 30 mM PDITC (p=0.02). There was no significant difference between the signal intensities of 20 mM and 30 mM of PDITC (p=0.06). Combining the differences in signal intensities, visual inspection by the naked eye, and the cost of the assay, 20 mM of PDITC was chosen for downstream work. The investigation of volume of PAMAM dendrimer in this method illustrates that the activation with 100 μL of dendrimer produced a stronger signal intensity than that of 80 μL PAMAM dendrimer in methanol (FIG. 6B, p=0.003).

In accordance with the principles herein, due to the three dimensional structure of a suitable carrier, such as FP, 1 μM size of beads and electrostatic repulsion between the negatively charged probe and target ssDNAs, the distance of spacer arm between tethered probes and surface of FP is necessary for the complement oligonucleotides to hybridize with each other efficiently on the active surface of FP. The spacer has two major functions: first, the maximum yield of probes can be immobilized on the FP surface. Second, the target ssDNAs can be kept in distance away from the FP surface to reduce the steric hindrance while hybridization. Six-carbon spacer and 12-carbon spacer were compared. Since 12-carbon spacer can provide a longer arm, as expected, 12-carbon spacer increases the signal intensities of targets after hybridization (FIG. 6C, p=0.02). It is also possible to further extend spacer length to enhance the detection sensitivity/efficiency of hybridization.

PAMAM dendrimers are hydrophilic. The activated effect with PAMAM dendrimers in methanol and in deionized water was compared. The signal intensity in water was stronger than that in methanol (FIG. 6D). There was no significant difference between the methods with dendrimers in the deionized water with distinct pH values (p=0.27). PAMAM dendrimer in water (pH 8.5) was selected for the follow-up work as it generated less background than that in water (pH 4.6) based on visual observation.

### Optimization of Amounts of Printed Probe, Target DNA and Volume of Magnetic Beads

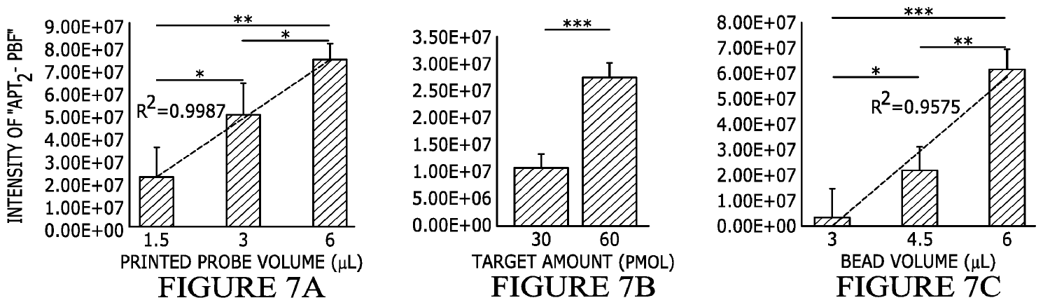

Once the method to activate the surface of FP was selected, the parameters of detection were optimized. The results achieved in accordance with the principles herein show that there is a potential to use even more probes, targets and beads for higher intensities of signals. Here, 120 μmol of printed probes, 60 μmol of target ssDNAs and 6 μL of magnetic beads were applied and resulted in the strongest signal intensities, respectively (FIGS. 7A, 7B and 7C). The filter paper used can provide larger surface space than that on planar platform. Moreover, this space is distributed in three dimensions with curved surfaces from FP fibers, which helps deduce electrostatic interactions amongst probes while immobilizing, and steric hindrance between probes and targets during hybridization. The relaxation of electronic repulsion facilitates higher density of aminated probes to be printed on the active surface and more target DNAs can be used for duplex formation. The superparamagnetic beads are with monolayer surface of streptavidin. Due to the high affinity of the streptavidin-biotin interaction, the beads can isolate biotinylated target ssDNAs and further carry out the specific detection of biotinylated target nucleic acid. The combination between 3D tethered probes on FP surface with 3D targets on the bead surfaces increases the option for duplex formation.

The controls of APT₂WO and TID qualitatively illustrated the background noise originated from the same sequence as probe, yet without modification of amino groups and the distinct probe sequence, but with amino groups modification. Both of printed areas in all cases did not present printed spots except for the activated condition with PAMAM dendrimer in deionized water (pH 4.6). The stricter wash condition would be required to increase the ratio of signal/noise after the step of amination with PAMAM dendrimer in water (pH 4.6).

Several methods were explored to activate the surface of FP. Considering the special structure of FP and hybridization efficiency at room temperature, the possible parameters were further optimized to increase sensitivity and specificity of the assay. Probe immobilization can be completed manually. These features enable the application of FP detection in any geographical distribution, as printing and detection do not require instrumentation. However, robotic printing can increase the density of immobilized probes and the printed area will be decreased so that both of the sensitivity and specificity in detection can be increased. In an embodiment, beads with 1 μM diameter served as an indicator of the presence of a target pathogen. Smaller beads can provide more curved surfaces than the larger ones in a given same surface space, thus the higher target-loading capacity will be achieved.

PAMAM dendrimer can further extend the surface area of filter paper, as it has three-dimensional microstructure. A method in accordance with the principles herein can include applying PAMAM dendrimer and PDITC or a composition including PAMAM dendrimer and PDITC configured to activate the filter paper. Further, devices and methods in accordance with the principles herein can provide a POC tool for rapid DNA detection.

TABLE 4
The sequences and their modifications of probes and target
in this study.

| | 5′ terminal | Carbon | Polythymine | | SEQ ID |
|---|---|---|---|---|---|
| | modification | spacer | spacer | Main sequences | NO: |
| APT | Amino group | C6 or | PolyT (15) | CGCATACCTCTCCAATCTCCG | 49 |
| | | C12 | | TTTACTGCACCTAATCACCT | |
| APT₂WO | None | C6 | PolyT (15) | CGCATACCTCTCCAATCTCCG | 50 |
| | | | | TTTACTGCACCTAATCACCT | |
| TID | Amino group | C6 | PolyT (15) | AAATTTGCCGACTCGCATAGG | 51 |
| | | | | TCTGTGATA | |
| RE-APT₂ | Biotin | None | None | AGGTGATTAGGTGCAGTAAAC | 52 |
| | | | | GGAGATTGGAGAGGTATGCG | |

TABLE 5
Effect Comparison of methanol and PAMAM dendrimer to activate the surface of
filter paper. A detailed description of the methods can be found in the main text.

| Method | Step 1 | Step 2 | Step 3 | Step 4 | Step 5 |
|---|---|---|---|---|---|
| (1) FP-GND | GA in DMP | NHS/DCC | | | |
| | | in DMF | | | |
| (2) 1% PAM-GND | 1% PAMAM | GA in DMF | NHS/DCC in | | |
| | in methanol | | DMF | | |
| (3) 3% PAM-GND | 3% PAMAM | GA in DMF | NHS/DCC in | | |
| | in methanol | | DMF | | |
| (4) 10% PAM-GND | 10% PAMAM | GA in DMF | NHS/DCC in | | |
| | in methanol | | DMF | | |
| (5) GND-1% PAM- | GA in DMP | NHS/DCC | 1% PAMAM | GA in DMF | NHS/DCC in |
| GND | | in DMF | in methanol | | in DMF |
| (6) GND-3% PAM- | GA in DMF | NHS/DCC | 3% PAMAM | GA in DMF | NHS/DCC in |
| GND | | in DMF | in methanol | | in DMF |
| (7) GND-10% PAM- | GA in DMF | NHS/DCC | 10% PAMAM | GA in DMF | NHS/DCC in |
| GND | | in DMF | in methanol | | in DMF |
| (8) GND-methanol- | GA in DMF | NHS/DCC | methanol | GA in DMF | NHS/DCC in |
| GND | | in DMF | | | in DMF |

TABLE 6
Comparison of active effect of APTS and PAMAM to activate the surface of
filter paper. A detailed description of the methods can be found in the main text.

| | Method | Step 1 | Step 2 | Step 3 | Step 4 | Step 5 | Step 6 |
|---|---|---|---|---|---|---|---|
| (1) | APTS-GND | APTS | GA in DMF | NHS/DCC | | | |
| | | | | in DMF | | | |
| (2) | APTS-GND- | APTS | GA in DMF | NHS/DCC | APTS | GA in DMF | NHS/DCC |
| | APTS-GND | | | in DMF | | | in DMF |
| (3) | APTS-PDI | APTS | PDITC in | | | | |
| | | | DMSO | | | | |
| (4) | APTS-PDI- | APTS | PDITC in | APTS | PDITC in | | |
| | APTS-PDI | | DMSO | | DMSO | | |
| (5) | APTS-PDI- | APTS | PDITC in | APTS | GA in DMF | NHS/DCC | |
| | APTS-GND | | DMSO | | | in DMF | |
| (6) | PAM-GND | PAMAM | GA in DMF | NHS/DCC | | | |
| | | | | in DMF | | | |
| (7) | PAM-GND- | PAMAM | GA in DMF | NHS/DCC | PAMAM | GA in DMF | NHS/DCC |
| | PAM-GND | | | in DMF | | | in DMF |
| (8) | PAM-PDI | PAMAM | PDITC in | | | | |
| | | | DMSO | | | | |
| (9) | PAM-PDI- | PAMAM | PDITC in | PAMAM | PDITC in | | |
| | PAM-PDI | | DMSO | | DMSO | | |
| (10) | PAM-PDI- | PAMAM | PDITC in | PAMAM | GA in DMF | NHS/DCC | |
| | PAM-GND | | DMSO | | | in DMF | |

TABLE 7
contemplated probes for detection of SARS-CoV-2

| Name | Description | Oligonucleotide Sequence (5′>3′) | SEQ ID NO: |
|---|---|---|---|
| 2019- | 2019- | GAC CCC AAA ATC AGC GAA AT | 53 |
| nCoV_N1-F | nCoV_N1 | | |
| | Forward | | |
| | Primer | | |
| 2019- | 2019- | TCT GGT TAC TGC CAG TTG AAT | 54 |
| nCoV_N1-R | nCoV_N1 | CTG | |
| | Reverse | | |
| | Primer | | |
| 2019- | 2019- | FAM-ACC CCG CAT TAC GTT TGG | 55 |
| nCoV_N1-P | nCoV_N1 | TGG ACC-BHQ1 | |
| | Probe | | |
| 2019- | 2019- | FAM-ACC CCG CAT /ZEN/ TAC GTT | 56 |
| nCoV_N1-P | nCoV_N1 | TGG TGG ACC-3IABkFQ | |
| | Probe | | |
| 2019- | 2019- | TTA CAA ACA TTG GCC GCA AA | 57 |
| nCoV_N2-F | nCoV_N2 | | |
| | Forward | | |
| | Primer | | |
| 2019- | 2019- | GCG CGA CAT TCC GAA GAA | 58 |
| nCoV_N2-R | nCoV_N2 | | |
| | Reverse | | |
| | Primer | | |
| 2019- | 2019- | FAM-ACA ATT TGC CCC CAG CGC | 59 |
| nCoV_N2-P | nCoV_N2 | TTC AG-BHQ1 | |
| | Probe | | |
| 2019- | 2019- | FAM-ACA ATT TGC /ZEN/ CCC CAG | 60 |
| nCoV_N2-P | nCoV_N2 | CGC TTC AG-3IABkF | |
| | Probe | | |
| RP-F | RNAse P | AGA TTT GGA CCT GCG AGC G | 61 |
| | Forward | | |
| | Primer | | |
| RP-R | RNAse P | GAG CGG CTG TCT CCA CAA GT | 62 |
| | Reverse | | |
| | Primer | | |
| RP-P | RNAse P | FAM-TTC TGA CCT GAA GGC TCT | 63 |
| | Probe | GCG CG-BHQ-1 | |
| RP-P | RNAse P | FAM-TTC TGA CCT /ZEN/ GAA GGC | 64 |
| | Probe | TCT GCG CG-3IABkFQ | |

In accordance with the principles herein, Point-of-care (POC) devices and methods therefore can be configured into a portable miniaturized device for rapid detection in a limited volume of sample, which simplifies the process and reduces the cost. To clinicians, POC testing usually aims for rapid and accurate diagnosis (e.g. of pathogens) in order to provide valuable information on the treatment and monitoring of diseases, especially to those with rapid progression (e.g. sepsis). POC devices according to the principles of the present disclosure provide on-site testing devices that can be practiced on bedside of patients instead of on the laboratory bench, thus it enables a rapid case management. The requirements of an ideal POC device usually are small size, portable, easy-to-use, cost efficient, and on-site visual detection.

Cellulose filter paper possesses some unique features suitable to POC testing for pathogens, for example, cellulose filter paper can be activated with chemicals for DNA immobilization, and its porous matrix enables a high density of immobilized probes and fluid transport in opposition to gravity without instrumentation, therefore a rapid analyte detection can be carried out on filter paper via capillary force. Furthermore, cellulose filter paper is cost efficient, abundant, portable, and easy-to-use.

To pathogen detection in early-stage of infectious diseases, nucleic acid testing (NAT) is reliable since antibody formation takes time, and NAT sensitivity is higher than the tests based on immune reactions. NAT assays are also more sensitive and faster than the tests based on cell culture, particularly for slow-growing or uncultured organisms. Broad range assay is one of NAT assays. 16S rRNA gene is a common target sequence for universal bacterial detection as it covers the conserved region of all bacteria.

In accordance with the principles herein, methods to activate the surface of cellulose filter paper and devices resulting therefrom are set forth. In accordance with the principles herein both synthesized oligonucleotides and bacterial DNA have been detected on the functionalized surface of filter paper configured in accordance with the methods set forth. The detection is carried out on basis of hybridization principle of complementary DNAs. The detective signals on the activated filter paper can be visible to the naked eye, performed on site, and can be analyzed quantitatively.

In accordance with the principles herein, suitable chemicals and indicator materials can be used to activate the filter paper, or carrier. For example, chemicals and materials suitable for the methods herein can include Glutaric anhydride (GA), N-hydroxysuccinimide (NHS), N,N′-Dicyclohexylcarbodiimide (DCC), N,N-dimethylformamide (DMF), Dimethyl sulfoxide (DMSO), 3-aminopropyltriethoxysilane (APTS), Whatman™ qualitative filter paper, SSC buffer and sodium dodecyl sulfate solution (SDS) (Sigma-Aldrich). DYNAL MyOne Dynabeads Streptavidin C1 (Fisher Scientific), DNA oligonucleotides (Sigma-Aldrich) and the custom-made magnetic stand (MagRach 16, Germany) were also applied.

### The Selection of Surface-Functionalization-System of Cellulose Filter Paper

Based on the chemicals of GA, NHS and DCC, six methods were evaluated to functionalize the surface of filter paper.

Conversion of Surface Groups into Carboxyl Groups on Filter Paper

There are hydroxyl groups on cellulose. Amino groups were produced by silyation with APTS (volume ratio of ethanol: H2O: APTS was 95:3:2) after 24 hours coating on filter paper at room temperature. Carboxyl groups were formed by the reaction of GA with hydroxyl or amino groups on filter paper overnight at room temperature. The reactions were done in saturated GA in DMF. Washing activated filter paper with DMF and deionized water and drying the filter paper at room temperature.

### Formation of NHS-Modified Filter Paper Surface

Filter paper with carboxyl surface was soaked into 1M NHS/1M DCC in DMF or DMSO for 4-5 hours while shaking and then was washed with DMF or DMSO and deionized water. In these reactions, the functional groups (—N═C═N—) in carbodimide compound (DCC) activated carboxyl groups to form an unstable intermediate; then NHS-modified filter paper surface was created via the reaction between NHS and this unstable intermediate. During this process, dicyclohexylurea byproduct was created thus careful wash was necessary. These were the principles of GA-NHS-DCC (GND, method 1) and APTS-GND (method 2).

### Fixation of Methanol and Recreation of NHS-Functional Filter Paper Surface

Following GND and APTS-GND, fixation via methanol (M) on filter paper was done in both methods, and the steps of GND were repeated on both surfaces. Those were the methods of GND-M-GND (method 3) and APTS-GND-M-GND (method 4). The principle of the method of M-GND (method 5) was to fix the surface of filter paper with methanol, and then followed by the steps of GND. After repeating the procedure of M-GND, the surface of M-GND-M-GND (method 6) was produced.

### Evaluation of Control's Position on Activated Filter Paper

To evaluate the control's position, four printing regions (position1-4, FIG. 3) in each functionalized paper slide were tested. Briefly, one region was for 20 μM aminated probe (APT₂), two were for DNA controls (20 μM unspecific aminated TID and APT₂W/O without amino group modification), and the last one was for 1×printing buffer (1×PBF) as a blank control.

The aminated probe (1.5 μL of 20 μM APT₂ in 1×PBF) was always printed in p1. The three controls (TID, APT₂W/O and 1×PBF) were immobilized in p2, 3 and 4 in different combinations. Finally, six different layouts for control immobilization were compared. The immobilization volume of each control was 1.5 μL. After incubation for 24 hours in a humid chamber, the surface groups of filter paper were blocked and filter paper was washed.

### Example 3: Visual Detection of Bacterial DNA Using Activated Paper Strip

Detection with Synthesized Target DNAs Bound with Magnetic Beads

Biotinylated target DNAs were bound with superparamagnetic beads coated with streptavidin. Fluid containing target DNAs transported the printed areas of filter paper via capillary force to carry out specific detection. The synthesized complementary probe and target were APT₂ and RE-APT₂. Both of the initiated amounts were 30 μmol. Six μL of beads were the start-up volume.

### Evaluation of Wash Effect

Five different procedures for filter paper washing were compared. Three methods started with buffer. They were washing at 55 degree for 10 minutes with either 4×SSC buffer containing 0.01% SDS (Method 1), or 2×SSC buffer containing 0.01% SDS (Method 2), or 1×SSC buffer containing 0.01% SDS (Method 5), followed by washing at room temperature for 1 minute with 0.4×SSC buffer or 0.2×SSC buffer or 0.1×SSC buffer, respectively, then deionized water was used to wash the corresponding filter paper slides for 1 minute at room temperature. The other two ways were to wash with deionized water either at 55 degree (Method 3) or at room temperature (Method 4) for 1 minute.

### Image Analysis

The MyImageAnalysis software was used to analyze signal intensities, and other suitable signal intensity software or devices can be used to evaluate reactions with the targets. The compared parameter of intensity was calculated by subtraction of volume of position 2 from that of position 1. Student's t-test was applied in statistical analysis, with significance level set to 0.05. The background signals from position 3 and 4 were used as qualitative parameters to evaluate the optional conditions, specifically for the detection by naked eye.

### Comparisons of Buffer to Dissolve the Printed Oligonucleotides and Spacer Length at 5 Terminal in Printed Oligonucleotides

Thirty pmol of probe and controls were printed on each active surface of filter paper in position 1, 2, 3 and 4. APT₂ was always dissolved in water, APT₂W/O and TID were dissolved in water and Tris buffer respectively. Six μL of magnetic beads and 30 μmol of RE-APT₂ were used in the detection on each activated filter paper. While comparing the different effects of carbon spacer (C6 and C12) at the 5 terminal end of printed oligonucleotides, 60 pmol of each probe and controls were printed on the filter paper. In the detection on each filter paper, 6 μL magnetic beads and 60 μmol RE-APT₂ were used.

### Optimization of Amounts of Printed Probe, Target and Beads Volume

In accordance with the principles herein, a method designed to optimize the parameters that can influence the hybridization effect was evaluated. In order to compare the amount of printed probe, three amounts of probe (1.5 μL, 3.0 μL, 6.0 μL of APT₂) were used on the functionalized filter paper, respectively. The amount of controls were the same on each same filter paper slide. Six A of magnetic beads and 30 μmol of RE-APT₂ were used in the detection. 60 μmol, 120 μmol and 240 μmol of target DNAs were compared to optimize the amount of target ssDNA, the amount of printed probe and magnetic beads for each filter paper detection were 120 μmol and 6.0 μL. 120 μmol of printed probe and 120 pmol of target DNAs were applied to optimize the volume of magnetic beads. Nine, 12 and 15 μL of magnetic beads were evaluated for the detection on each paper slide.

Comparison of Solvents that NHS/DCC was Dissolved in

When preparing the creation of ester NHS-modified filter paper surface, two solvents (DMSO and DMF) were used to dissolve NHS/DCC. Three concentrations were compared (1 M, 500 mM and 250 mM). All the other steps to activate filter paper were the same as the earlier work. Six A of probes and controls were printed respectively on each active filter paper. For each paper slide detection, 6.0 μL of magnetic beads and 60 μmol of RE-APT₂ were used.

### Bacteria DNA Detection on Basis of Broad-Range Primers

In order to detect DNA in accordance with the principles herein, firstly, bacterial amplicons binding to beads via interaction between biotin and streptavidin were formed. The amplicons were produced via broad range 16S rRNA gene PCR with forward primer 64F (Bio-BGYCTWANRCATGCAAGTYG (SEQ ID NO: 65), reverse primer 803R (CTACCRGGGTATCTAATCC (SEQ ID NO: 17) and campylobacter DNA template. PCR conditions were: 400 nM primers, 1×Kapa PCR mix (Kapa Biosystems), 1.6 μl template in a 20 μl reaction. Tubes were incubated in a thermocycler at 95° C. for 3 min, then cycled at 95° C. for 30 sec, 50° C. for 30 sec, and 72° C. for 30 sec for 45 times. 0.1 M NaOH was applied to denature double-strand DNAs (dsDNAs) into single-strand DNAs (ssDNAs) followed by washing with 1×PBS-T. ssDNAs labeled with biotin were dissolved in 1×PBS-T for detection.

Bacterial probe (CTACCAGGGTATCTAATCCTGTTTGCTCCCCACGCTTTCG (SEQ ID NO: 66)) was part of amplicons with the same modification and spacer as RE-APT₂ at 5 terminal. Two hundred fifty mM, 500 mM and 1M of NHS/DCC in DMSO were evaluated under this condition. The effect of different printed volumes (6 μL, 8 μL and 10 μL) of the probe (20 μM) on the results of detection was compared on the activated filter paper.

### Surface-Activation-System of Cellulose Filter Paper

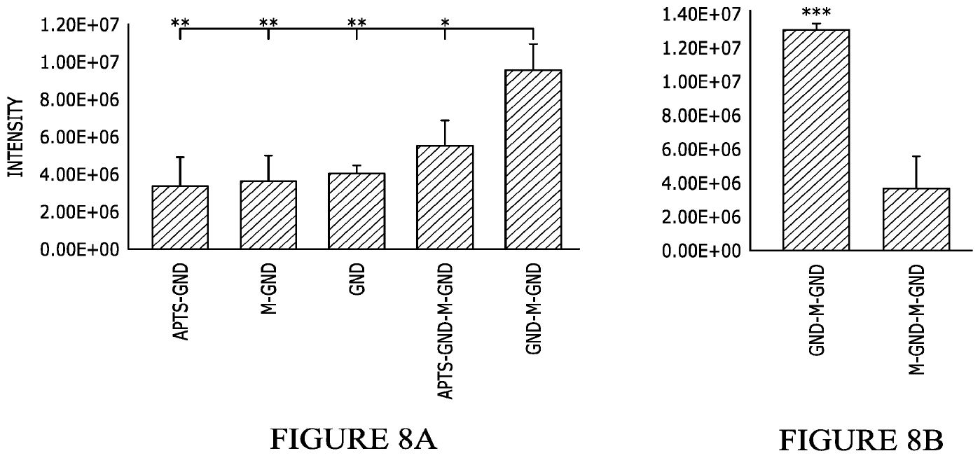

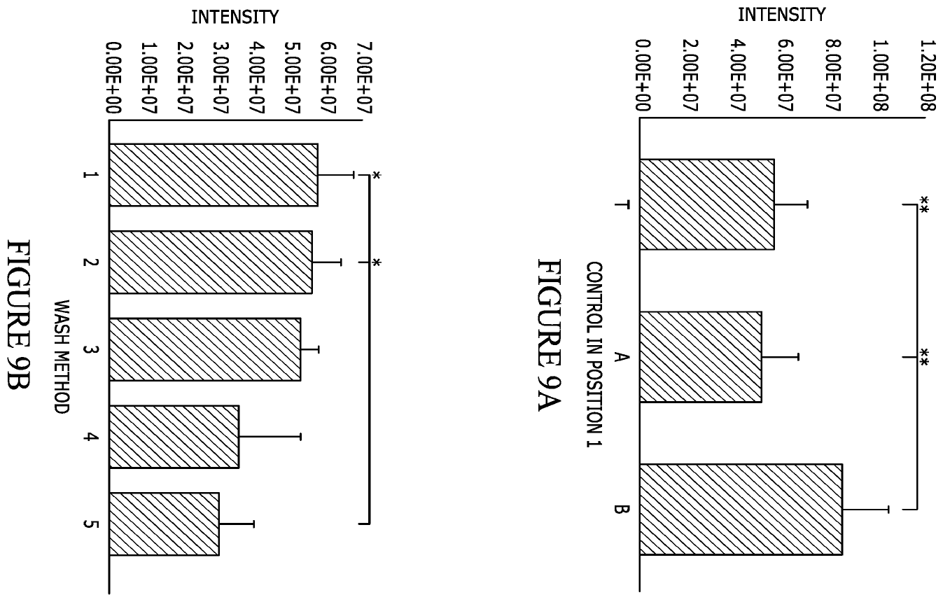

GA, DCC, and NHS can activate glass slide with functional carboxylic ester surface of NHS step by step, which avoids the cross-linking phenomenon that happens to homo-bifunctional groups e.g. PDITC. We discovered that the functional ester groups were also produced on the surface of filter paper, and fixed via methanol. Following fixation, repetition of the formation of the ester groups of NHS on the same filter paper can increase the signal intensity and homogeneity (FIG. 8). The reason is that methanol can preserve fibers and their dimensions in filter paper and further retain the functional groups on filter paper while washing. GND-M-GND was the method chosen for downstream work as it resulted in the highest signal-to-noise ratio (FIG. 8).

### Layout for Specific Probe Detection on Activated Filter Paper

The factors that interfere with the target signal intensity include physical absorbing of oligonucleotides to the surface of filter paper, electrostatic repulsion between bases, and an immediate event of hybridization at room temperature (1 minute-2 minute). Aiming at avoiding cross-reactivity, we used three distinct controls, namely TID, APT₂W/O and 1×PBF. The controls of TID and APT₂W/O were synthetic oligonucleotides, part of them was same as probe APT₂, either modification or sequence. As the solution that the probe was dissolved in, 1×PBF was a blank control for the investigation.

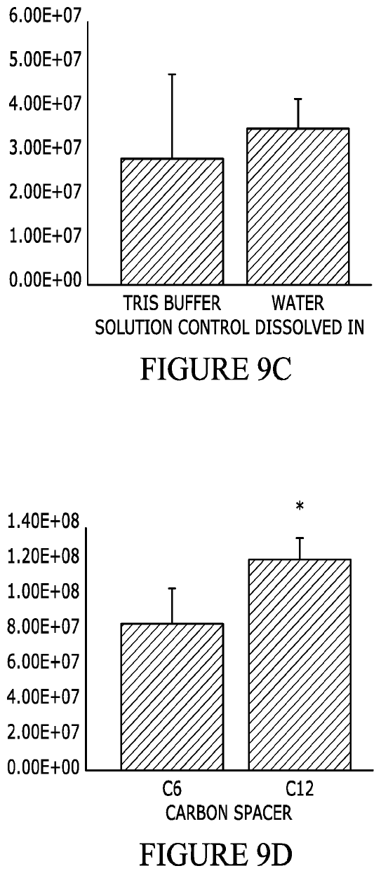

The strongest background was produced in position 2, the difference of intensity between that of position 1 and 2 was compared under distinct situations. The result displayed the best signal intensity was created from the groups that blank control (B) was printed in position 2 instead of TID (T) and APT₂W/O (A) (FIG. 9A). The unspecific oligonucleotides in position 2 should increase the background noise due to the effects of physical adsorption and oligonucleotide cross-reactivity. This was verified by our results and the strongest noise was created by the groups that the control APT₂WO was immobilized in position 2 (FIG. 9A). Significant difference in signal intensity was shown between the groups with PBF and TID in position 2 (p=0.009) as well as the groups with PBF and APT₂W/O in same position (p=0.005). There was no significant difference between the groups with TID and APT₂W/O in position 2, respectively (p=0.266) (FIG. 9A). Targets were printed 1×PBF in position 2,TID in position 3 and APT₂W/O in position 4 for downstream work.

### Evaluation of Wash Method

To provide reliable detection, it is essential to increase the ratio of signal/noise in diagnostic systems. It includes increasing target signal intensity, while removing the unspecific signal and decreasing the background. Optimization of wash methods is one of the options to increase the specific ratio of signal/noise. The highest signal-to-noise ratio resulted from methods of 1 and 2 (FIG. 9B, p=0.44). Significant difference was shown between methods of 1 and 5 (p=0.014), also between 2 and 5 (p=0.013). Therefore, method 2 was selected for the subsequent work.

### Evaluations of the Solution Printed Oligonucleotides Dissolved in and Carbon Spacer

Tris(hydroxymethyl)aminomethane (Tris) contains a primary amine that can occupy the NHS-ester groups on surface of filter paper. The detection effect was compared while the control ssDNAs (APT₂W/O and TID) were dissolved in Tris buffer and water individually. The result displayed that the signal intensity with controls dissolved in water was stronger than that in Tris buffer (FIG. 9C) though the difference was not significant (p=0.3). Thus, water was chosen to dissolve the oligonucleotides. Since carbon spacer could influence the hybridization efficiency, a stronger hybridized signal between specific target and probe has been created in the methods herein with longer carbon spacer modification at 5 terminal of target ssDNA (p=0.027) (FIG. 9D).

### Evaluations of Printed Probe Amount, Target DNA Amount and Magnetic Bead Volume

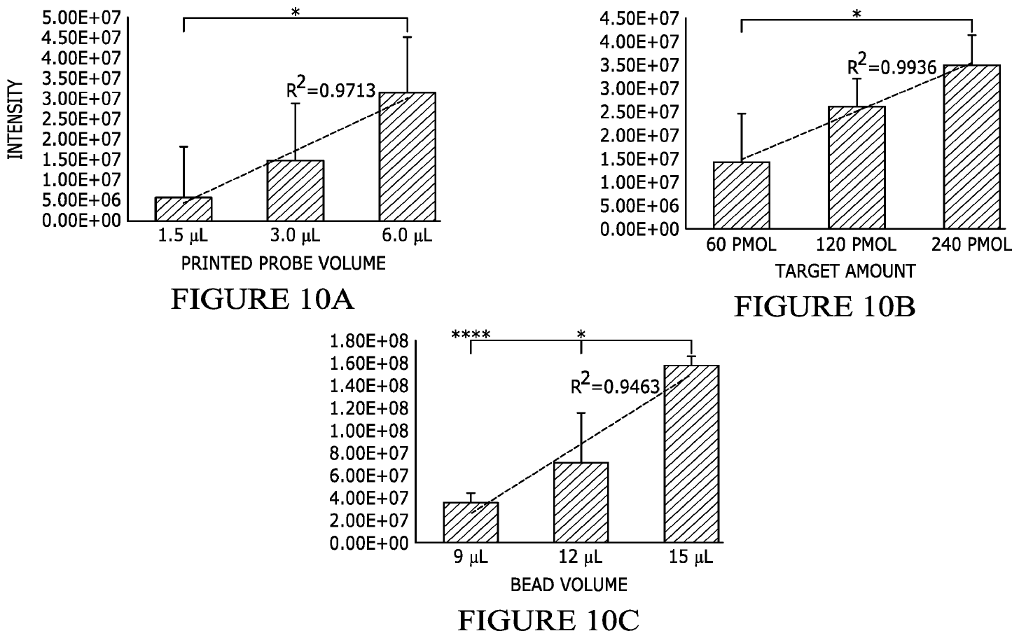

Considering efficacy and specificity of hybridization, we accessed the different amounts of printed probe and target DNA, and different volumes of magnetic bead under the current functionalization system. The results displayed that higher amounts of printed probe, target DNA and larger volume of bead produced stronger signal intensities (FIGS. 10A, 10B and 10C), and presented a tendency of linear increase. The porous matrix of filter paper provides larger surface comparing with the traditional platform of microarray for probe immobilization, and this feature enables a high amount of specific target DNA detection in a short time (1 min-2 min) at room temperature, particularly with a high amount of printed probe and a large volume of beads. The pore size of filter paper and the diameter of magnetic beads can be optimized to determine the flow rate of target solution through filter paper in order to achieve higher ratio of signal/noise.

The target oligonucleotides bind super-parallel magnetic beads due to the strong interaction between biotin modifying the oligonucleotides and streptavidin on the surface of magnetic particles. The brown color originated from the irons of magnetic particles played a role of a signal indicator for naked eye detection and a basis of quantitative analysis.

### Evaluations of Solvent Dissolving NHS/DCC and Solvent Concentration

DMSO would be less toxic than DMF, therefore in the process of NHS-ester surface formation on filter paper, the functionalized effect on filter paper with different solvents was compared: DMF and DMSO, which NHS/DCC was dissolved in. During this procedure, a higher amount of dicyclohexylurea byproduct was produced in higher concentration of NHS/DCC in polar solvents, specifically in DMF. The isourea byproduct is water insoluble and makes filter paper wash difficult. Thus, we chose 250 mM NHS/DCC in DMSO to produce NHS-ester surface of filter paper for synthetic oligonucleotides detection.

### Evaluation of Bacterial Detection

Routine diagnostic methods detecting bacteria are either based on culture, or on pre-designed strain-specific probes. However, many bacteria cannot be cultured and NAT assays have a limited multiplexing ability due to cross-reactivity of probes. Based on our previous study, the pair of primers of 64F and 803R targeting the 16S rRNA gene was selected for their lack of cross-reactivity with human genome. That was the pilot result for the detection of bacterial genomic DNA on our activated filter paper and on-site signals were visible without wash (Movie S1). Considering the degenerated primers were used, the parameters of probe immobilization and functionalization were optimized.

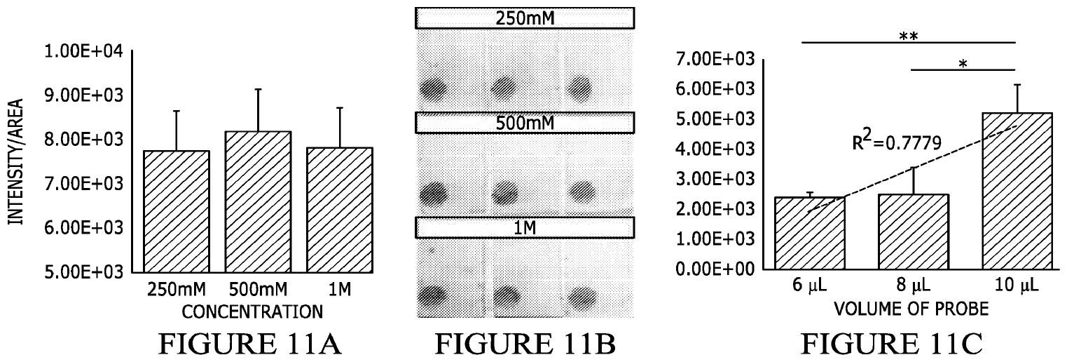

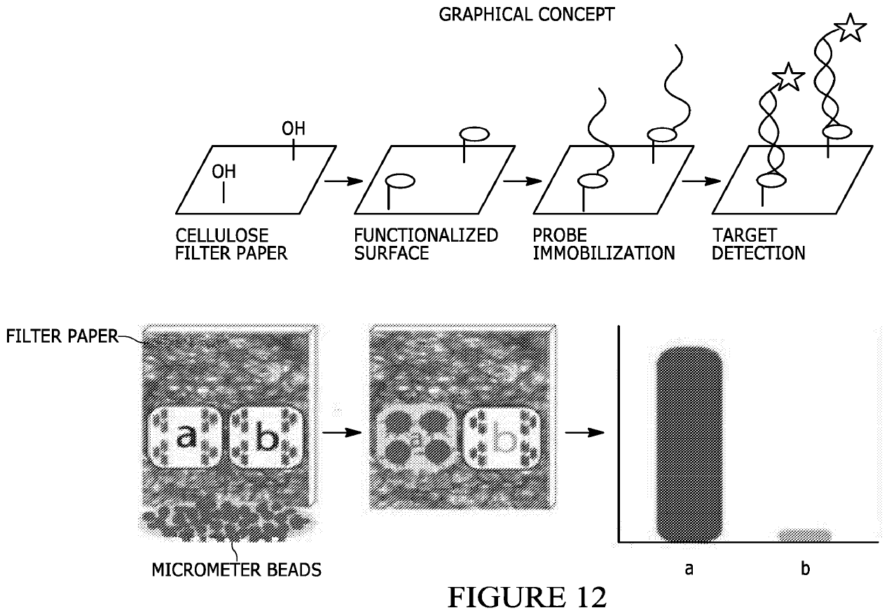

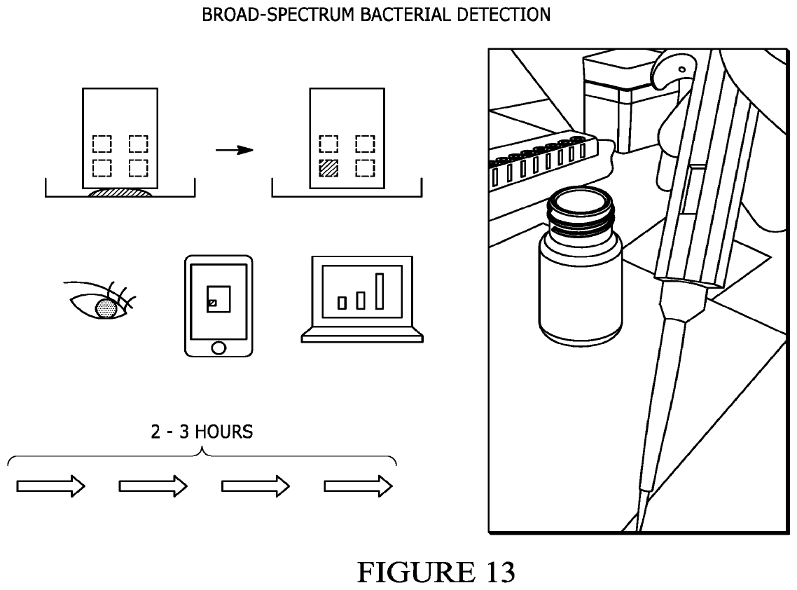

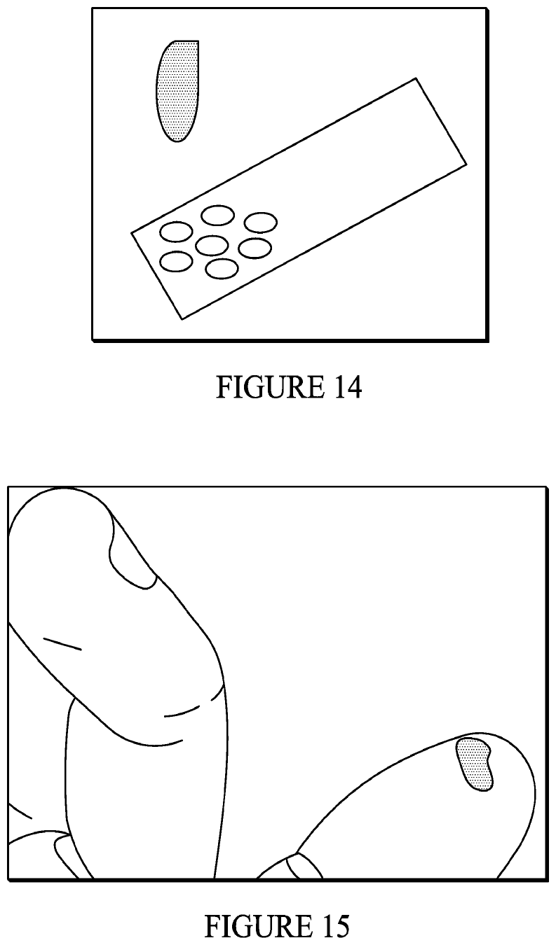

The higher volume of printed probe produced stronger intensity (FIG. 11C). The activated system with 500 mM of NHS/DCC in DMSO provided the clearest signals though there was no significant difference between the different concentrations in the effects of detection (FIG. 11A). Lower concentration of NHS/DCC (250 mM) led to stronger background, however higher concentration of NHS/DCC produced more byproducts (FIG. 11B), which increased the difficulty in wash while the fibers being maintained. Therefore, the concentration of 500 mM of NHS/DCC in DMSO was chosen based on visual signals on site by naked eye and fiber stability of filter paper. Twenty μL of PCR product was used in each detection. Density (Intensity/Area) of signals in this section were compared.

Cellulose filter paper is an ideal support in POC testing for DNA detection mainly due to its porous matrix. We have developed a novel chemistry surface on the surface of filter paper that is suitable for DNA detection in POC testing (FIGS. 12-13). The reactions between GA, NHS and DCC produces carboxyl ester surface on filter paper enabling NH₂-DNA detection. After methanol fixation on filter paper, more functional groups are created via the same reactions, thus a higher signal intensity is captured. As a proof of concept of this activated filter paper, on-site visual detection of bacteria was shown, which highlights the developed method in this study. In accordance with the principles herein, POC devices can be customized and provide a solution to pathogen detection in a number of settings. Also, since antibiotics do not work in fungal and viral infection, POC devices described herein can enable faster clinical decisions with small sample volumes (FIGS. 14-15). Systems constructed in accordance with the principles herein can be configured to detect all bacteria and all fungi in blood samples; identify universal marker genes; and specific probes for the most common pathogens in BSI.

### Embodiments

Embodiment 1: A point of care (POC) diagnostic device comprising:
a single fibrous carrier configured to receive and transport a fluid sample to one or more embedded capture probes, each of the one or more embedded capture probes configured to visually display one or more outputs indicating rapid universal detection of bacteria and/or fungi and/or viruses, and one or more specific target bacteria and/or one or more specific target fungi and/or one or more specific viruses present in the fluid sample.
Embodiment 2: A POC diagnostic device as recited in embodiment 1, the fibrous carrier further defined by a filter paper.
Embodiment 3: A POC diagnostic device as recited in embodiment 1, the fluid sample further defined by a small sample size in the range of 0.01 ml-0.5 ml.
Embodiment 4: A POC diagnostic device as recited in embodiment 1, wherein the fluid sample includes at least one of blood, urine, saliva, breast milk, mucus, pus, sweat, tears, CSF, semen, secretions, serum, plasma or bronchoalveolar lavage fluid.
Embodiment 5: A POC diagnostic device as recited in embodiment 1, wherein the fluid sample contains a bodily fluid.
Embodiment 6: A POC diagnostic device as recited in embodiment 1, wherein the fluid sample contains fluid used in the manufacturing of pharmaceutical or food products.
Embodiment 7: A POC diagnostic device as recited in embodiment 6, wherein the fluid sample is bottled water.
Embodiment 8: A POC diagnostic device as recited in embodiment 1, wherein the fluid sample contains metalworking fluid, coolant or potable water.
Embodiment 9: A POC diagnostic device as recited in embodiment 1, configured to identify antimicrobial resistance genes in the fluid sample.
Embodiment 10: A POC diagnostic device as recited in embodiment 3, wherein DNA and/or protein components in the fluid sample are detectable and identifiable from the fluid sample received via the single fibrous carrier.
Embodiment 11: A POC diagnostic device as recited in embodiment 1, further comprising one or more embedded capture probes each capture probe configured to display a visual output indicating rapid detection of antimicrobial resistance genes.
Embodiment 12: A POC diagnostic device as recited in embodiment 3, wherein antimicrobial resistance genes in the fluid sample are detectable and identifiable from the fluid sample received via the single fibrous carrier.
Embodiment 13: A POC diagnostic device as recited in embodiment 3, wherein antimicrobial resistance genes in the fluid sample are detectable and identifiable from a smaller fluid sample than currently required to detect antimicrobial resistance genes in the laboratory.
Embodiment 14: A POC diagnostic device as recited in embodiment 1, the one or more outputs each having a variation in intensity based on levels of the one or more specific target bacteria or the one or more specific target fungi or the one or more specific target viruses present in the fluid sample, such that an image of the single fibrous carrier can indicate categories and quantity of pathogens, which can further guide treatment of a fluid sample source.
Embodiment 15: A POC diagnostic device as recited in embodiment 1, the one or more outputs each having a variation in intensity based on levels of the one or more specific target bacteria or the one or more specific target fungi or the one or more specific target viruses present in the fluid sample, such that an image of the single fibrous carrier can indicate categories and quantity of pathogens in a human patient or a sick animal, which can further guide treatment and case management of the patient or the sick animal.
Embodiment 16: A POC diagnostic device as recited in embodiment 1, the one or more outputs providing an indication of an intensity level of a bacterial and/or viral concentration in the fluid sample.
Embodiment 17: A POC diagnostic device as recited in embodiment 1, the one or more outputs generated via a chemical reaction between DNA amplicons originating from the fluid sample and beads.
Embodiment 18: A POC diagnostic device as recited in embodiment 1, the one or more outputs generated via a chemical reaction between DNA amplicons originating from the fluid sample and color generating components.
Embodiment 19: A POC diagnostic device as recited in embodiment 1, further comprising an orientation component to aide in confirming the location of the one or more embedded capture probes on the single fibrous carrier.
Embodiment 20: A POC diagnostic device as recited in embodiment 1, wherein the location of the one or more embedded capture probes on the single fibrous carrier is determined by the position of the one or more embedded capture probes on the single fibrous carrier.
Embodiment 21: A POC diagnostic device as recited in embodiment 19, the orientation component comprising to at least one of printed text and other indicia.
Embodiment 22: A POC diagnostic device as recited in embodiment 20, the location identifiable via an offset to the paper.
Embodiment 23: A POC diagnostic device as recited in embodiment 1, the one or more embedded capture probes further comprising activateable treatments embedded in the one or more embedded capture probes that release in response to rapid detection of the one or more specific target bacteria and/or the one or more specific target fungi and/or the one or more specific target viruses present in the fluid sample.
Embodiment 24: A filter paper functionalized by one of the following: FP-GND; FP-1% PAM-GND; FP-3% PAM-GND; FP-10% PAM-GND; FP-GND-1% PAM-GND; FP-GND-3% PAM-GND; FP-GND-10% PAM-GND; FP-GND-methanol-GND; FP-APTS-GND; FP-APTS-GND-APTS-GND; FP-APTS-PDITC; FP-APTS-PDITC-APTS-PDITC; FP-APTS-PDITC-APTS-GND; FP-PAMAM-GND; FP-PAMAM-GND-PAMAM-GND; FP-PAMAM-PDITC; FP-PAMAM-PDITC-PAMAM-PDITC; FP-PAMAM-PDITC-PAMAM-GND; FP-methanol-GND; FP-methanol-GND-methanol-GND.
Embodiment 25: A fibrous carrier functionalized by one of the following: FP-GND; FP-1% PAM-GND; FP-3% PAM-GND; FP-10% PAM-GND; FP-GND-1% PAM-GND; FP-GND-3% PAM-GND; FP-GND-10% PAM-GND; FP-GND-methanol-GND; FP-APTS-GND; FP-APTS-GND-APTS-GND; FP-APTS-PDITC; FP-APTS-PDITC-APTS-PDITC; FP-APTS-PDITC-APTS-GND; FP-PAMAM-GND; FP-PAMAM-GND-PAMAM-GND; FP-PAMAM-PDITC; FP-PAMAM-PDITC-PAMAM-PDITC; FP-PAMAM-PDITC-PAMAM-GND; FP-methanol-GND; FP-methanol-GND-methanol-GND.
Embodiment 26: A POC diagnostic device as recited in embodiment 1, each of the one or more embedded capture probes functionalized by at least one of the following: FP-GND; FP-1% PAM-GND; FP-3% PAM-GND; FP-10% PAM-GND; FP-GND-1% PAM-GND; FP-GND-3% PAM-GND; FP-GND-10% PAM-GND; FP-GND-methanol-GND; FP-APTS-GND; FP-APTS-GND-APTS-GND; FP-APTS-PDITC; FP-APTS-PDITC-APTS-PDITC; FP-APTS-PDITC-APTS-GND; FP-PAMAM-GND; FP-PAMAM-GND-PAMAM-GND; FP-PAMAM-PDITC; FP-PAMAM-PDITC-PAMAM-PDITC; FP-PAMAM-PDITC-PAMAM-GND; FP-methanol-GND; FP-methanol-GND-methanol-GND.
Embodiment 27: A diagnostic kit configured to generate an output displaying cross-reactivity of a pathogen biomarker and/or to analyze and/or confirm presence of a universal and/or specific pathogen within a fluid sample, the fluid sample identified/detected/confirmed by at least one of FP-GND; FP-1% PAM-GND; FP-3% PAM-GND; FP-10% PAM-GND; FP-GND-1% PAM-GND; FP-GND-3% PAM-GND; FP-GND-10% PAM-GND; FP-GND-methanol-GND; FP-APTS-GND; FP-APTS-GND-APTS-GND; FP-APTS-PDITC; FP-APTS-PDITC-APTS-PDITC; FP-APTS-PDITC-APTS-GND; FP-PAMAM-GND; FP-PAMAM-GND-PAMAM-GND; FP-PAMAM-PDITC; FP-PAMAM-PDITC-PAMAM-PDITC; FP-PAMAM-PDITC-PAMAM-GND; FP-methanol-GND; FP-methanol-GND-methanol-GND.
Embodiment 28: The diagnostic kit as recited in embodiment 27, the fluid sample analyzable and/or transportable via the point of care device of embodiment 1.
Embodiment 29: A test strip configured to generate a visual output in the presence of a universal pathogen, based on detection of universal biomarkers of genes and/or proteins and selection of universal pairs of primers without cross-reactivity with human genomic DNA and/or a specific pathogen based on detection of specific markers of genes and/or proteins and selection of specific pairs of primers without cross-reactivity with human genomic DNA identified/detected/confirmed by at least one of FP-GND; FP-1% PAM-GND; FP-3% PAM-GND; FP-10% PAM-GND; FP-GND-1% PAM-GND; FP-GND-3% PAM-GND; FP-GND-10% PAM-GND; FP-GND-methanol-GND; FP-APTS-GND; FP-APTS-GND-APTS-GND; FP-APTS-PDITC; FP-APTS-PDITC-APTS-PDITC; FP-APTS-PDITC-APTS-GND; FP-PAMAM-GND; FP-PAMAM-GND-PAMAM-GND; FP-PAMAM-PDITC; FP-PAMAM-PDITC-PAMAM-PDITC; FP-PAMAM-PDITC-PAMAM-GND; FP-methanol-GND; FP-methanol-GND-methanol-GND.
Embodiment 30: A universal or specific pathogen biomarker for genes and/or proteins identifiable from fluid samples identified/detected/confirmed by at least one of FP-GND; FP-1% PAM-GND; FP-3% PAM-GND; FP-10% PAM-GND; FP-GND-1% PAM-GND; FP-GND-3% PAM-GND; FP-GND-10% PAM-GND; FP-GND-methanol-GND; FP-APTS-GND; FP-APTS-GND-APTS-GND; FP-APTS-PDITC; FP-APTS-PDITC-APTS-PDITC; FP-APTS-PDITC-APTS-GND; FP-PAMAM-GND; FP-PAMAM-GND-PAMAM-GND; FP-PAMAM-PDITC; FP-PAMAM-PDITC-PAMAM-PDITC; FP-PAMAM-PDITC-PAMAM-GND; FP-methanol-GND; FP-methanol-GND-methanol-GND.
Embodiment 31: A rapid process for identifying pathogens and generated samples of genes and proteins in a fluid sample comprising a testing media functionalized by at least one of FP-GND; FP-1% PAM-GND; FP-3% PAM-GND; FP-10% PAM-GND; FP-GND-1% PAM-GND; FP-GND-3% PAM-GND; FP-GND-10% PAM-GND; FP-GND-methanol-GND; FP-APTS-GND; FP-APTS-GND-APTS-GND; FP-APTS-PDITC; FP-APTS-PDITC-APTS-PDITC; FP-APTS-PDITC-APTS-GND; FP-PAMAM-GND; FP-PAMAM-GND-PAMAM-GND; FP-PAMAM-PDITC; FP-PAMAM-PDITC-PAMAM-PDITC; FP-PAMAM-PDITC-PAMAM-GND; FP-methanol-GND; FP-methanol-GND-methanol-GND.
Embodiment 32: A method of targeting the 16S rRNA gene comprising the steps of: optimizing a selection of PCR primers to reduce cross-reaction with human DNA, thereby increasing specificity and sensitivity for BSI and other pathogen diagnosis; and configuring a capture probe using a selected PCR primer on a selected media to receive a fluid sample from a fluid source at the probe.
Embodiment 33: The method as recited in embodiment 32, further comprising the step of adding a reactive component to the selected PCR primer, wherein 16S rRNA gene present in the fluid sample reacts with the reactive component on or within the selected media to generate a reactive output, such as a visual output, thereby providing rapid and accurate pathogen detection for rare bacterial DNA in blood or other fluids, even in the presence of abundant host DNA.
Embodiment 34: The method as recited in embodiment 33, the target identified/detected/confirmed with at least one of FP-GND; FP-1% PAM-GND; FP-3% PAM-GND; FP-10% PAM-GND; FP-GND-1% PAM-GND; FP-GND-3% PAM-GND; FP-GND-10% PAM-GND; FP-GND-methanol-GND; FP-APTS-GND; FP-APTS-GND-APTS-GND; FP-APTS-PDITC; FP-APTS-PDITC-APTS-PDITC; FP-APTS-PDITC-APTS-GND; FP-PAMAM-GND; FP-PAMAM-GND-PAMAM-GND; FP-PAMAM-PDITC; FP-PAMAM-PDITC-PAMAM-PDITC; FP-PAMAM-PDITC-PAMAM-GND; FP-methanol-GND; FP-methanol-GND-methanol-GND.
Embodiment 35: A method of targeting the 16S rRNA gene comprising the steps of: optimizing the selection of PCR primers, wherein the selection of the PCR primers increases specificity and sensitivity for pathogen detection, priorly using at least one of FP-GND; FP-1% PAM-GND; FP-3% PAM-GND; FP-10% PAM-GND; FP-GND-1% PAM-GND; FP-GND-3% PAM-GND; FP-GND-10% PAM-GND; FP-GND-methanol-GND; FP-APTS-GND; FP-APTS-GND-APTS-GND; FP-APTS-PDITC; FP-APTS-PDITC-APTS-PDITC; FP-APTS-PDITC-APTS-GND; FP-PAMAM-GND; FP-PAMAM-GND-PAMAM-GND; FP-PAMAM-PDITC; FP-PAMAM-PDITC-PAMAM-PDITC; FP-PAMAM-PDITC-PAMAM-GND; FP-methanol-GND; FP-methanol-GND-methanol-GND; and configuring the capture probe to provide reactive regions for each of the selected PCR primers in the presence of a fluid sample.
Embodiment 36: A system configured to produce a rapid, clear, regular and visible output signal that observable via the naked eye, and analyzable quantitatively to indicate bacterial detection on a capture probe, such as by using 16S rDNA probes on an activated paper surface as the capture probe for universal bacterial diagnosis.
Embodiment 37: A system configured to embed a selection of PCR primers on a capture probe, further configured to generate an output indicating presence of the 16S rRNA gene, wherein the system can be optimized to avoid cross-reaction with human or animal DNA background in samples derived from or containing human or animal fluid.
Embodiment 38: The system as recited in embodiment 37, the system providing rapid and accurate pathogen detection for rare bacterial DNA in blood in the presence of abundant host DNA and also increasing the specificity and sensitivity for BSI diagnosis.
Embodiment 39: A Point-of-care (POC) detection device comprising: polyamidoamine (PAMAM) dendrimer, and p-Phenylene diisothiocyanate (PDITC), configured on a filter paper in order to activate the surface of filter paper to bind DNA molecules from a sample containing DNA.
Embodiment 40: A POC device as recited in embodiment 39, formed by a process comprising the steps of primary amination of the surface of filter paper with PAMAM dendrimer, followed by creating isothiocyanate groups via PDITC, and subsequently repeating these two steps.
Embodiment 41: The POC device as recited in embodiment 39, the filter paper formed of a highly porous structure, such that multiple printed probes, target DNAs and magnetic beads can be embedded therein and provide high signal intensities in the detection area via probe/target duplex formation.
Embodiment 42: The POC device as recited in embodiment 39, configured to carry out a rapid, specific and cost-efficient DNA detection, wherein the filter paper is further defined by cellulose filter paper.
Embodiment 43: The POC device as recited in embodiment 39 further comprising embedded treatment materials, the device configured to connect a sample to the capture probe(s) for diagnosis and to selectively release treatment for an infectious disease, the device configured to further facilitate identification of antimicrobial drug resistance genes based on a small sample size.
Embodiment 44: A point of care device (POC) comprising: surface-functionalization systems of cellulose filter paper including glutaric anhydride, N-hydroxysuccinimide, N, N′-Dicyclohexylcarbodiimide and methanol.
Embodiment 45: The POC device as recited in embodiment 44, configured to identify synthetic oligonucleotides and bacterial genomic DNA.
Embodiment 46: A filter paper comprising a universal or a specific capture probe configured to selectively bind to a DNA segment of a pathogen present in a sample labeled with superparamagnetic beads and to display a visual signal when so bound due to an iron color produced by the superparamagnetic beads.
Embodiment 47: The filter paper as recited in embodiment 46, wherein targets bound with the superparamagnetic beads are collectable and purifiable via a magnetic stand.

We claim:

1. A point of care (POC) diagnostic device for identifying a target nucleic acid sequence in a sample, comprising:
a fibrous carrier comprising functionalized fibers;
one or more capture probes bound to one or more of the functionalized fibers in one or more first discrete locations of the fibrous carrier, the one or more capture probes being capable of selectively binding with the target nucleic acid sequence;
one or more control probes bound to one or more of the functionalized fibers in one or more second discrete locations of the fibrous carrier; and
one or more indicia disposed on the fibrous carrier to identify the first and second discrete locations.

2. The device of claim 1, wherein the fibrous carrier is filter paper.

3. The device of claim 2, wherein the filter paper is cellulose filter paper.

4. The device of claim 2 or 3, wherein the filter paper is highly porous.

5. The device of any one of claims 1 to 4, wherein the functionalized fibers comprise a nucleic acid binding moiety.

6. The device of claim 5, wherein the one or more capture probes comprise nucleic acids.

7. The device of claim 6, wherein the one or more capture probes comprise a synthetic oligonucleotide, genomic DNA, or genomic RNA.

8. The device of claim 7, wherein the one or more capture probes comprise bacterial genomic DNA.

9. The device of any one of claims 1 to 8, wherein the one or more capture probes comprise primers capable of amplifying a bacterial genomic DNA.

10. The device of claim 9, wherein the primers are capable of amplifying a bacterial genomic DNA that does not cross-react with a human genomic DNA.

11. The device of claim 9 or 10, wherein the one or more capture probes comprises a primer capable of amplifying a 16S rRNA gene.

12. The device of claim 11, wherein the one or more capture probes is a primer selected from 64F, 363F, 520F, 530F, 806R, 1027R, and 1100R.

13. The device of any one of claims 1 to 12, wherein the one or more capture probes selectively binds with a target nucleic acid sequence from a bacterium, a virus, a fungus, or combinations thereof.

14. The device of claim 13, wherein the one or more capture probe selectively binds DNA of a bacterium.

15. The device of claim 14, wherein the bacterium is one or more of Staphylococcus aureus, Escherichia coli, and Campylobacter jejuni.

16. The device of claim 15, wherein the one or more capture probes selectively binds DNA and/or RNA of viruses.

17. The device of claim 16, wherein the one or more capture probes selectively binds one or more of RNA of SARS-CoV-2, influenza viruses, and Cytomegalovirus.

18. The device of any one of claims 1 to 17, comprising a visual label that binds to the target nucleic acid sequence.

19. A method for making the device of any one of claims 1 to 18, comprising the steps of:
(a) treating the fibrous carrier with one or more reagents to functionalize fibers of the fibrous carrier with a nucleic acid binding moiety;
(b) binding the one or more capture probes to the one or more of the functionalized fibers in the one or more first discrete locations of the fibrous carrier, the nucleic acid binding moiety binding the capture probe to the one or more functionalized fibers; and
(c) applying one or more control probes to one or more second discrete locations of the fibrous carrier.

20. The method of claim 19, wherein the nucleic acid binding moiety is an active ester, thiocarbamate, or isothiocyanate.

21. The method of claim 20, wherein the nucleic acid binding moiety is an active thiocarbamate formed by reacting the fibrous carrier with polyamidoamine (PAMAM) dendrimer and p-phenylene diisothiocyanate (PDITC).

22. The method of claim 20, wherein the nucleic acid binding moiety is an active ester formed by reacting the fibrous carrier with glutaric anhydride, N-hydroxysuccinimide, N, N′-dicyclohexylcarbodiimide (GND), or glutaric anhydride, N-hydroxysuccinimide, N, N′-dicyclohexylcarbodiimide and methanol (GND-methanol).

23. The method of any one of claims 19 to 22, wherein with the one or more reagents comprises GND; 1% PAM followed by GND; 3% PAM followed by GND; 10% PAM followed by GND; GND followed by 1% PAM-GND; GND followed by 3% PAM followed by GND; GND followed by 10% PAM followed by GND; GND followed by methanol followed by GND; APTS followed by GND; APTS followed by GND followed by APTS followed by GND; APTS followed by PDITC; APTS followed by PDITC followed by APTS followed by PDITC; APTS followed by PDITC followed by APTS followed by GND; PAMAM followed by GND; PAMAM followed by GND followed by PAMAM followed by GND; PAMAM followed by PDITC; PAMAM followed by PDITC followed by PAMAM followed by PDITC; PAMAM followed by PDITC followed by PAMAM followed by GND; methanol followed by GND; or methanol followed by GND followed by methanol followed by GND.

24. A method for detecting the presence of a pathogen in a sample, the method comprising:
(a) contacting the sample comprising or suspected of comprising a target nucleic acid sequence from the pathogen with a visual label under conditions to bind the visual label to a target nucleic acid sequence thereby providing a labeled sample;
(b) contacting the device of any one of claims 1 to 18 with the labeled sample, wherein upon contact the target nucleic acid sequence if present binds to at least one capture probe of the one or more of the capture probes in at least one of the one or more first discrete locations congregating the visual label attached to the nucleic acid sequence of the pathogen in the at least one of the one or more first discrete locations, thereby generating a visual output; and
(c) washing the device to remove unbound portions of the sample.

25. The method of claim 24, further comprising pre-treating the sample prior to labeling the sample.

26. The method of claim 25, wherein pre-treating the sample comprises extraction of a target nucleic acid from the sample, amplification by PCR, and/or denaturation of double stranded DNA.

27. The method of any one of claims 24 to 26, wherein the pathogen is a bacterium, a virus, or a fungus.

28. The method of any one of claims 24 to 27, wherein the pathogen is a bacterium.

29. The method of any one of claims 24 to 28, wherein the sample is a fluid sample.

30. The method of any one of claims 24 to 29, wherein the sample comprises a bodily fluid.

31. The method of claim 30, wherein the sample comprises blood, urine, saliva, breast milk, mucus, pus, sweat, tears, cerebrospinal fluid (CSF), semen, serum, plasma, or bronchoalveolar lavage fluid, or combinations thereof.

32. The method of any one of claims 24 to 31, wherein the sample comprises fluid used in the manufacturing of pharmaceutical or food products.

33. The method of any one of claims 24 to 31, wherein the sample comprises bottled water.

34. The method of any one of claims 24 to 31, wherein the sample comprises metalworking fluid, coolant or potable water.

35. The method of any one of claims 24 to 34, wherein the volume of the sample is about 0.01 mL to about 0.5 mL.

36. The method of any one of claims 24 to 35, wherein the nucleic acid is genomic DNA or genomic RNA.

37. The method of claim 36, wherein the nucleic acid is genomic DNA.

38. The method of claim 37, wherein the genomic DNA comprises a 16S rRNA gene.

39. The method of claim 38, wherein the genomic DNA comprises an antimicrobial resistance gene.

40. The method of any one of claims 24 to 39, wherein the sample is from an animal, and wherein the capture probe selectively binds the target nucleic acid and/or target protein and does not bind animal DNA.

41. The method of any one of claims 24 to 40, wherein an intensity of the visual output indicates the quantity of a pathogen in a sample.

42. The method of any one of claims 24 to 40, wherein the visual output indicates the quantity and identity of a pathogen in a sample.

43. The method of claim 41 or 42, wherein the visual output indicates the quantity, identity, or both quantity and identity of a pathogen based on a variation in intensity of the visual output.

44. The method of any one of claims 24 to 43, wherein the visual label comprises superparamagnetic beads.

45. The method of claim 44, wherein the superparamagnetic beads selectively bind to the target nucleic acid and/or target protein.

46. A kit for determining the presence of a pathogen in a sample comprising:
a detection device, comprising:
a fibrous carrier comprising functionalized fibers; and
one or more capture probes bound to one or more of the functionalized fibers in one or more first discrete locations of the fibrous carrier, the one or more capture probes being capable of selectively binding with the target nucleic acid sequence; and
one or more control probes bound to one or more of the functionalized fibers in one or more second discrete locations of the fibrous carrier;
a visual label for labeling the sample; and
instructions for labeling a sample with the visual label and contacting the sample with the detection device to transport the sample through the fibrous carrier and expose the target nucleic acid sequence, if present, to the one or more capture probes.

47. The kit of claim 46, wherein the fibrous carrier is filter paper.

48. The kit of claim 47, wherein the filter paper is cellulose filter paper.

49. The kit of claim 47 or 48, wherein the filter paper is highly porous.

50. The kit of any one of claims 46 to 49, wherein the functionalized fibers comprise a nucleic acid binding moiety.

51. The kit of any one of claims 46 to 50, wherein the capture probe is a nucleic acid.

52. The kit of claim 51, wherein the capture probe comprises synthetic oligonucleotides, genomic DNA, or genomic RNA.

53. The kit of claim 52, wherein the capture probe selectively binds with a target nucleic acid sequence from bacterial genomic DNA.

54. The kit of any one of claims 46 to 53, wherein the capture probe comprises a primer which amplifies a bacterial genomic DNA.

55. The kit of claim 54, wherein the primer amplifies a bacterial genomic DNA without cross-reacting with a human genomic DNA.

56. The kit of claim 54 or 55, wherein the capture probe comprises a primer which amplifies a 16S rRNA gene.

57. The kit of claim 56 wherein the capture probe is a primer selected from 64F, 363F, 520F, 530F, 806R, 1027R, and 1100R.

58. The kit of any one of claims 46 to 57, wherein the one or more capture probes selectively binds DNA or RNA of a bacterium, a virus, a fungus, or combinations thereof.

59. The kit of claim 58, wherein the one or more capture probes selectively binds DNA of a bacterium.

60. The kit of claim 59, wherein the bacterium is one or more of Staphylococcus aureus, Escherichia coli, and Campylobacter jejuni.

61. The kit of claim 60, wherein the one or more capture probes selectively binds DNA or RNA of a virus.

62. The kit of claim 61, wherein the one or more capture probes selectively binds RNA of SARS-CoV-2, influenza viruses, or Cytomegalovirus.

63. The kit of claim 60, wherein the one or more capture probes selective binds DNA of fungi.

64. The kit of claim 63, wherein the one or more capture probes selectively binds DNA of Aspergillus or Candida.

65. The kit of any one of claims 46 to 64, wherein the visual label comprises superparamagnetic beads.

66. The kit of claim 65, wherein the superparamagnetic beads selectively bind to the target nucleic acid and/or target protein.
